(19) United States
(12) Patent Application Publication
(10) Pub. No.: US 2017/0106031 A1
(43) Pub. Date: Apr. 20, 2017

(54) CONPOSITIONS AND METHODS FOR THE PREVENTION AND TREATMENT OF CANCER

(71) Applicant: UNIVERSITY OF OTTAWA, Ottawa (CA)

(72) Inventors: Chantal MATAR, Ottawa (CA); Tri VUONG, Ottawa (CA)

(73) Assignee: UNIVERSITY OF OTTAWA, Ottawa, ON (CA)

(21) Appl. No.: 14/783,637

(22) PCT Filed: Apr. 11, 2014
(86) PCT No.: PCT/CA2014/050372

§ 371 Date: Oct. 9, 2015

Related U.S. Application Data
(60) Provisional application No. 61/811,076, filed on Apr. 11, 2013.

(51) Int. Cl. A61K 36/45; C12P 1/04; A23L 33/105; A61K 9/00

(52) U.S. Cl. CPC A61K 36/45; A61K 9/0095; A61K 9/0053; C12P 1/04; A23L 33/105; A23V 2002/00

(57) ABSTRACT

Compositions comprising biotransformed fruit extracts and methods of using the compositions in the prevention and treatment of cancer are provided. The fruit extracts are biotransformed by fermentation with a bacterial strain having all the identifying characteristics of Serratia vaccinii. The compositions can be used to inhibit tumour growth, tumour metastasis and cancer stem cell (CSC) development.

### FIELD OF THE INVENTION

The present invention relates to the field of cancer and, in particular, to the use of compositions comprising bacterially fermented fruit extracts in the prevention and treatment of cancer.

### BACKGROUND OF THE INVENTION

Cancer is a broad group of various diseases, all involving unregulated cell growth. In cancer, cells divide and grow uncontrollably, forming malignant tumours, and invade nearby parts of the body. The cancer may also spread to more distant parts of the body through the lymphatic system or bloodstream. There are over 200 different known cancers that afflict humans.

Tumours are sustained in their pathological growth by a small subpopulation of tumour cells with “stem-like” properties. These cells are reportedly responsible for resistance in both chemotherapy and radiation (Wicha M. S., et al., 2006, Cancer Res., 66(4):1883-90 [discussion 95-6]; Wicha M. S., 2006, Clin Cancer Res., 12(19):5606-7). This subset of cancer cells in breast cancer displays increased ability to self-renew and reproduce breast cancer heterogeneity and are designated Cancer Stem Cells (CSCs). CSCs are a highly tumorigenic cell type and have been hypothesized to be key drivers of cancer (Graziano A, et al., 2008, J Cell Biochem., 103(2):408-12). In breast cancer, CSCs have the ability to grow as spheres or mammospheres known as cancer stem cell phenotype CD44+/CD24 low (Wicha M. S., 2006, Breast Cancer Res., 8(5):109; Dontu G, et al., 2003, Cell Prolif., 36 Suppl 1:59-72). In breast cancer, disease progression and relapse after therapy and tumour removal has been linked to the chemoresistance of CSCs (Lee H. E., et al., 2011, Br J Cancer., 104(11):1730-8; Korkaya H, et al., 2011, Clin Cancer Res., 17(19):6125-9; Iliopoulos D, et al., 2009, Sci Signal., 2(92):ra62). In prostate cancer, CSCs have the ability to grow as spheres or prostatospheres (Iliopoulos, D., et al., 2011, Cancer Res, 71(9):3196-201). This subset of cells is often related to increased prostate cancer invasion and chemoresistance (Yu, C., et al., 2012, Minerva Urol Nefrol, 64(1):19). In addition, selective targeting of skin cancer stem cells is now proposed as a strategy in cancer cell elimination in melanoma skin cancer types (Shmidt et al., 2011, Oncotarget, 2(4):313-20).

The blueberry fruit is rich in phenolic compounds such as hydroxycinnamic acids, flavonoids and proanthocyanins. Fermentation of blueberries with a novel strain of bacteria, Serratia vaccinii, isolated from the blueberry flora increases the phenolic content and antioxidant activity of juice from the blueberries (International Patent Application Publication No. WO2004/101770; Martin, L. and Matar, C., 2005, J Sci Food Agri, 85:1477-1484). The fermented blueberry juice also exhibits modified biological activity, for example demonstrating inhibition of nitric oxide production in macrophages (Vuong, T., et al., 2006, Journal of Food Biochemistry, 30:249-268), increased antiobesity and antidiabetic effects (U.S. Patent Application Publication No. 2010/0092583; Vuong, T., et al., 2007, Can J Physiol Pharmacol, 85: 956-965; Vuong, T., et al., 2009, Int J Obes (Lond), 33: 1166-1173), and protection of neurons against hydrogen peroxide-induced oxidative stress (Vuong, T., et al., 2010, Br J Nutr, 104: 656-663).

Bioactives from vegetal biomass such as polyphenols from blueberry have been shown to inhibit growth and metastatic potential of breast cancer cells through modulation of phosphatidylinositol 3-kinase (PI3K), an important component of the IL-6 signalling pathway (Adams, L. S., et al., 2010, Cancer Res, 70: 3594-3605; Faria, A., et al., 2010, Phytother Res, 24:1862-1869), and phenolic extracts from European blueberries have been shown to inhibit proliferation of, and induce apoptosis in, breast cancer cells (Nguyen, V., et al., 2010, J Med Food, 13:278-285).

Sera from mice that were lifetime exposed to a diet supplemented with blueberry powder have been shown to repress mammosphere formation of MCF-7 and MDA-MB-231 breast cancer cells in vitro (Montales, M. T., et al., 2012, Carcinogenesis, 33: 652-660). A mixture of isolated blueberry phenolic compounds, however, were only active in MDA-MB-231 breast cancer cells and increasing the dosage of the phenolic compounds resulted in a loss of activity.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to compositions and methods for the prevention and treatment of cancer. In one aspect, the invention relates to a composition comprising a biotransformed fruit extract for use to inhibit cancer stem cell development in a subject, wherein the biotransformed fruit extract is prepared by fermenting a fruit extract with a bacterial strain having all the characteristics of Serratia vaccinii.

In another aspect, the invention relates to a composition comprising a biotransformed fruit extract for use to inhibit cancer metastasis in a subject, wherein the biotransformed extract is prepared by fermenting a fruit extract with a bacterial strain having all the characteristics of Serratia vaccinii.

In another aspect, the invention relates to a composition comprising a biotransformed fruit extract for use as a complementary therapy for the treatment of cancer in a subject undergoing an anti-cancer treatment, wherein the biotransformed extract is prepared by fermenting a fruit extract with a bacterial strain having all the characteristics of Serratia vaccinii.

In another aspect, the invention relates to a composition comprising a biotransformed fruit extract for use in cancer chemoprevention in a subject, wherein the biotransformed fruit extract is prepared by fermenting a fruit extract with a bacterial strain having all the characteristics of Serratia vaccinii.

In another aspect, the invention relates to a composition comprising a biotransformed fruit extract for use to inhibit activation of the IL-6 pathway in a cancer cell, wherein the biotransformed extract is prepared by fermenting a fruit extract with a bacterial strain having all the characteristics of Serratia vaccinii.

In another aspect, the invention relates to a composition comprising a biotransformed fruit extract for use to inhibit expression of miR-210 miRNA in a cancer cell, wherein the biotransformed extract is prepared by fermenting a fruit extract with a bacterial strain having all the characteristics of Serratia vaccinii.

In another aspect, the invention relates to a use of a biotransformed fruit extract in the manufacture of a composition for inhibiting cancer stem cell development in a subject, wherein the biotransformed fruit extract is prepared by fermenting a fruit extract with a bacterial strain having all the characteristics of Serratia vaccinii.

In another aspect, the invention relates to a use of a biotransformed fruit extract in the manufacture of a composition for inhibiting cancer metastasis in a subject, wherein the biotransformed extract is prepared by fermenting a fruit extract with a bacterial strain having all the characteristics of Serratia vaccinii.

In another aspect, the invention relates to a use of a biotransformed fruit extract in the manufacture of a composition for complementary cancer therapy in a subject undergoing an anti-cancer treatment, wherein the biotransformed extract is prepared by fermenting a fruit extract with a bacterial strain having all the characteristics of Serratia vaccinii.

In another aspect, the invention relates to a use of a biotransformed fruit extract in the manufacture of a composition for reducing the risk of cancer in a subject, wherein the biotransformed fruit extract is prepared by fermenting a fruit extract with a bacterial strain having all the characteristics of Serratia vaccinii.

In another aspect, the invention relates to a use of a biotransformed fruit extract in the manufacture of a composition for inhibiting activation of the IL-6 pathway in a cancer cell, wherein the biotransformed extract is prepared by fermenting a fruit extract with a bacterial strain having all the characteristics of Serratia vaccinii.

In another aspect, the invention relates to a use of a biotransformed fruit extract in the manufacture of a composition for inhibiting expression of miR-210 miRNA in a cancer cell, wherein the biotransformed extract is prepared by fermenting a fruit extract with a bacterial strain having all the characteristics of Serratia vaccinii.

In another aspect, the invention relates to a method of inhibiting cancer stem cell development in a subject comprising administering to the subject an effective amount of a composition comprising a biotransformed fruit extract, wherein the biotransformed fruit extract is prepared by fermenting a fruit extract with a bacterial strain having all the characteristics of Serratia vaccinii.

In another aspect, the invention relates to a method of inhibiting cancer metastasis in a subject comprising administering to the subject an effective amount of a composition comprising a biotransformed fruit extract, wherein the biotransformed extract is prepared by fermenting a fruit extract with a bacterial strain having all the characteristics of Serratia vaccinii.

In another aspect, the invention relates to a method of complementary therapy for the treatment of cancer comprising administering to a subject undergoing an anti-cancer treatment an effective amount of a composition comprising a biotransformed fruit extract, wherein the biotransformed extract is prepared by fermenting a fruit extract with a bacterial strain having all the characteristics of Serratia vaccinii.

In another aspect, the invention relates to a method of decreasing the risk of a subject developing cancer comprising administering to the subject an effective amount of a composition comprising a biotransformed fruit extract, wherein the biotransformed extract is prepared by fermenting a fruit extract with a bacterial strain having all the characteristics of Serratia vaccinii.

In another aspect, the invention relates to a method for inhibiting activation of the IL-6 pathway in a cancer cell comprising contacting the cancer cell with a composition comprising a biotransformed fruit extract, wherein the biotransformed extract is prepared by fermenting a fruit extract with a bacterial strain having all the characteristics of Serratia vaccinii.

In another aspect, the invention relates to a method for inhibiting expression of miR-210 miRNA in a cancer cell comprising contacting the cancer cell with a composition comprising a biotransformed fruit extract, wherein the biotransformed extract is prepared by fermenting a fruit extract with a bacterial strain having all the characteristics of Serratia vaccinii.

In certain embodiments, the cancer is brain cancer, breast cancer, colon cancer, ovarian cancer, pancreatic cancer, prostate cancer, liver cancer, bladder cancer, melanoma or multiple myeloma. In some embodiments, the cancer is breast cancer, prostate cancer or melanoma.

In certain embodiments, the fruit extract is from one or more berries from the genus Vaccinium. In some embodiments, the fruit extract comprises a blueberry extract.

In certain embodiments, the biotransformed fruit extract is prepared by fermenting a medium comprising the fruit extract with the bacterial strain at a temperature of between about 8° C. and about 36° C. and a pH of about pH3.3 to about pH5.0, for between about 1 day and about 12 days.

In certain embodiments, the biotransformed fruit extract is prepared by fermenting the fruit extract with the bacterial strain and a yeast, such as Saccharomyces cerevisae.

In certain embodiments, the biotransformed fruit extract is characterized as having a total phenolic content at least two times greater than non-biotransformed fruit extract.

In certain embodiments, the composition is administered orally to the subject.

In certain embodiments, the composition is formulated as a pharmaceutical composition, a nutraceutical, a dietary supplement, a cosmetic preparation, a functional food or a beverage.

### BRIEF DESCRIPTION OF THE DRAWINGS

These and other features of the invention will become more apparent in the following detailed description in which reference is made to the appended drawings.

FIG. 1 depicts the proliferation of 4T1 (A), MDA-MB-231 (B), and MCF-7 (C) breast cancer cells after treatment with either 150 or 200 μM GAE (Gallic Acid Equivalent) of either biotransformed blueberry juice (BBJ) or normal blueberry juice (NBJ) for 24 h. All values are means of 3 separate experiments±SEM. * denotes statistical significance at p<0.05 vs. control.

FIG. 2 depicts cell mobility and invasion of 4T1 (A), MDA-MB-231 (B), and MCF-7 (C) cells after treatment with 100 μM GAE (Gallic Acid Equivalent) of either biotransformed blueberry juice (BBJ) or normal blueberry juice (NBJ) for 24 or 48 h, and cell invasion of 4T1 (D) and MDA-MB-231 (E) cells after treatment with either 100 or 150 μM GAE of BBJ or NBJ for 24 h. All values are means of 3 separate experiments±SEM. * denotes statistical significance at p<0.05 vs. control.

FIG. 3 depicts mammosphere formation of 4T1 (A), MDA-MB-231 (B), and MCF-7 (C) cells after treatment with either 100 or 150 μM GAE (Gallic Acid Equivalent) of either biotransformed blueberry juice (BBJ) or normal blueberry juice (NBJ) for 4-7 days. All values are means of 4 separate experiments±SEM. * denotes statistical significance at p<0.05 vs. control.

FIG. 4 depicts IL-6 production by 4T1 (A), MDA-MB-231 (B), and MCF-7 (C) cells after treatment with 150 μM GAE (Gallic Acid Equivalent) of either biotransformed blueberry juice (BBJ) or normal blueberry juice (NBJ) for 6 h or 24 h. All values are means of 4 separate experiments±SEM. * denotes statistical significance at p<0.05 vs. control.

FIG. 5 depicts the phosphorylation of STAT3, PI3K, PDK1, and PTEN in 4T1 (A-D), MDA-MB-231 (E-H) and MCF-7 (I-L) mammospheres after treatment with 150 μM GAE (Gallic Acid Equivalent) of either biotransformed blueberry juice (BBJ) or normal blueberry juice (NBJ) for 6 h. All values are means of 3 separate experiments±SEM. * denotes statistical significance at p<0.05 vs. control.

FIG. 6 depicts the phosphorylation of ERK1/2, MAPK p38, and JNK in 4T1 (A-C), MDA-MB-231 (D-F) and MCF-7 (G-I) mammospheres after treatment with 150 μM GAE (Gallic Acid Equivalent) of either biotransformed blueberry juice (BBJ) or normal blueberry juice (NBJ) for 2 h. All values are means of 3 separate experiments±SEM. * denotes statistical significance at p<0.05 vs. control.

FIG. 7 depicts tumour volume (A), tumour weight (B), and mammosphere formation (C) for tumours in mice that received a 2-week pretreatment and a 3-week post-inoculation treatment with either biotransformed blueberry juice (BBJ) or normal blueberry juice (NBJ) incorporated in drinking water (a final 12.5%, 25%, and 50% v/v blueberry juice solution). All values are means of 2 separate experiments±SEM (n=16). * denotes statistical significance at p<0.05 vs. control.

FIG. 8 depicts metastasis in lungs of mice that received a 2-week pretreatment and a 3-week post-inoculation treatment with either biotransformed blueberry juice (BBJ) or normal blueberry juice (NBJ) incorporated in drinking water (a final 12.5%, 25%, and 50% v/v blueberry juice solution). All values are means of 2 separate experiments±SEM (n=8). * denotes statistical significance at p<0.05 vs. control.

FIG. 9 presents the 16S rRNA gene sequence of the bacterium Serratia vaccinii [SEQ ID NO:1].

FIG. 10 depicts the proliferation of B16F10 (A) and HS294t (B) cells after treatment with either 100 or 150 μM GAE (Gallic Acid Equivalent) of either biotransformed blueberry juice (BBJ) or normal blueberry juice (NBJ) for 24 h. All values are means of 3 separate experiments±SEM. * denotes statistical significance at p<0.05 vs. control.

FIG. 11 depicts the spheroid formation of B16F10 (A) and HS294t (B) cells after treatment with 100 or 150 μM GAE (Gallic Acid Equivalent) of either biotransformed blueberry juice (BBJ) or normal blueberry juice (NBJ) for 5 days. All values are means of 3 separate experiments±SEM. * denotes statistical significance at p<0.05 vs. control.

FIG. 12 depicts expression of selected microRNA by 4T1 cells after a 24 hour treatment with 60 mEG/ml of biotransformed blueberry juice (BBJ) or normal blueberry juice (NBJ) by qRT-PCR. All values are means±SEM. * denotes statistical significance at p<0.05 vs. control. *** p<0.001

FIG. 13 depicts prostatosphere formation of LNCaP (A) and Du145 (B) cells after treatment with either biotransformed blueberry juice (BBJ) or normal blueberry juice (NBJ) for 5 days. All values are means of 3 separate experiments±SEM. * denotes statistical significance at p<0.05 vs. control.

FIG. 14 depicts phosphorylation of STAT3 (A), Akt (B) and p70S6K (C) in LNCaP spheroids after treatment with 150 μM GAE (Gallic Acid Equivalent) of either biotransformed blueberry juice (BBJ) or normal blueberry juice (NBJ) for 24 h, and phosphorylation of ERK1/2 (D), MAPKp38 (E), and JNK (F) in LNCaP spheroids after treatment with 150 μM GAE (Gallic Acid Equivalent) of either biotransformed blueberry juice (BBJ) or normal blueberry juice (NBJ) for 2 h.

### DETAILED DESCRIPTION OF THE INVENTION

The present invention relates to compositions comprising biotransformed fruit extracts and methods of using the compositions in the prevention and treatment of cancer.

As demonstrated herein, an exemplary biotransformed fruit extract is capable of influencing components of key signaling pathways involved in tumour invasion and metastasis, and in the development of cancer stem cells (CSCs) in various types of cancer. The biotransformed fruit extract was also demonstrated to inhibit tumour growth, tumour metastasis and CSC development in various cancers. CSCs are a highly tumorigenic cell type, which in clinical settings are believed to be responsible for relapse, therapy resistance and tumour recurrence.

Accordingly, in certain embodiments, the invention relates to methods of inhibiting cancer metastasis in a subject, for example metastasis of primary tumours, by administration of a composition comprising a biotranformed fruit extract. In certain embodiments, the invention relates to methods of inhibiting cancer stem cell development in a subject by administration of a composition comprising a biotranformed fruit extract. In some embodiments, the invention relates to methods of delaying or preventing the recurrence of a cancer in a subject by administration of a composition comprising a biotranformed fruit extract. In some embodiments, the invention relates to methods of treating therapy-resistant cancers, such as drug-resistant cancers, by administration of a composition comprising a biotransformed fruit extract. In some embodiments, the invention relates to methods of delaying or preventing the progression of a cancer in a subject by administering a composition comprising a biotransformed fruit extract. In some embodiments, the invention relates to methods of improving the efficacy of conventional cancer therapies, such as chemotherapy or radiation, by administering to a subject a composition comprising a biotransformed fruit extract. In some embodiments, the invention relates to methods of using a compositions comprising a biotransformed fruit extract in adjunct therapy for the treatment of cancer.

Certain embodiments of the invention relate to the use of the compositions for cancer chemoprevention, for example, to lower the risk of developing cancer or to slow cancer development.

Use of the compositions comprising one or more biotransformed fruit extracts as a complementary cancer therapy to traditional therapies is also provided in certain embodiments. For example, the compositions may be provided as a nutraceutical or health supplement to be taken by a cancer patient in order to reduce the likelihood of cancer stem cell development, metastasis or progression, or for cancer chemoprevention.

By “biotransformed” it is meant that the fruit extracts are fermented with a bacterial strain having all the identifying characteristics of Serratia vaccinii, as described in International Patent Application Publication No. 2004/101770, under conditions as described herein. In certain embodiments, a biotransformed fruit extract may have been fermented with a bacterial strain having all the identifying characteristics of Serratia vaccinii in combination with a yeast, for example, Saccharomyces cerevisae.

### DEFINITIONS

Unless defined otherwise, all technical and scientific terms used herein have the same meaning as commonly understood by one of ordinary skill in the art to which this invention belongs.

As used herein, the term “aggressive cancer” refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types.

A subject “suspected of having an aggressive cancer,” refers to a patient who has a tumour or lesion, which tumour or lesion has features correlated with the development of advanced disease, for example, markers predictive of aggressive disease. In a specific example, an indication of aggressive breast cancer is a tumour that is estrogen-receptor negative (ER−). Alternatively, the tumour may be ER positive, but the patient may exhibit other markers predictive of aggressive disease, such as node positivity. In these situations adjuvant therapies may be applied.

A “recurrent cancer,” cancer “recurrence” or “relapse” refers to a cancer that has recurred (come back), usually after a period of time during which the cancer could not be detected. The cancer may come back to the same place as the original (primary) tumour or to another place in the body.

The terms “therapy” and “treatment,” as used interchangeably herein, refer to an intervention performed with the intention of alleviating the symptoms associated with, preventing the development of, or altering the pathology of a disease. Thus, the terms therapy and treatment are used in the broadest sense, and in various embodiments include one or more of the prevention (prophylaxis), moderation, reduction, and/or curing of a disease at various stages. Those in need of therapy/treatment thus may in various embodiments include those already having the disease, as well as those prone to, or at risk of developing, the disease, and those in whom the disease is to be prevented.

The term “adjunct therapy” or “adjunctive therapy” refers to a treatment used together with a primary treatment in order to assist the primary treatment.

Administration of a composition described herein “in combination with” one or more further therapies is intended to include simultaneous (concurrent) administration and consecutive administration. Consecutive administration is intended to encompass various orders of administration of the therapy/ies and the composition to the subject with administration of the therapy/ies and the composition being separated by a defined time period that may be short (for example in the order of minutes) or extended (for example in the order of days or weeks).

The term “nutraceutical,” as used herein, refers to a food or dietary supplement that protects or promotes health and/or provides a benefit to a subject which affects the health of the subject.

The term “inhibit” and grammatical variations thereof, as used herein, means to reduce, halt or hold in check, and thus inhibition may be complete or partial and may be of short or long term duration. The term may be used in the context of inhibiting a process or action already begun or it may be used in the context of inhibiting initiation of a process or action.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to.

### Compositions

The compositions in accordance with the invention comprise one or more biotransformed fruit extracts. The compositions are typically prepared as liquids, but may optionally be subsequently treated by conventional techniques to convert them into an alternate form, such as a solid, semi-solid, powder or the like.

### Bacteria

Bacteria suitable for use to prepare biotransformed fruit extracts for inclusion in the compositions described herein are described in International Patent Application Publication No. 2004/101770. A representative bacterium, which was isolated from the microflora of lowbush blueberry (Vaccinium angustifolium), was deposited with the International Depository Authority of Canada (IDAC), Bureau of Microbiology, Health Canada, 1015 Arlington St., Winnipeg, Manitoba, Canada on Jan. 16, 2003 under Accession Number 160103, and named Serratia vaccinii. Analysis of the biochemical profile and partial sequence of the 16S rRNA gene of Serratia vaccinii indicates that it belongs to the family Enterobacteriaceae.

Bacteria suitable for use to biotransform fruit extracts for inclusion in the compositions described herein have the same identifying characteristics as the Serratia vaccinii bacterium deposited under Accession Number 160103. Serratia vaccinii is characterised in that it is a gram negative, catalase positive, facultatively anaerobic coccobacillus with a fermentative metabolism. Serratia vaccinii can ferment a number of sugars, including D-glucose, D-fructose, D-mannose, arbutin, esculin, salicin, saccharose and D-raffinose, but does not ferment L-arabinose. Under certain conditions, Serratia vaccinii can also ferment mannitol, lactose and trehalose as described in International Patent Application Publication No. 2004/101770. Serratia vaccinii produces acetoin, hydrolyses hippurate and produces a number of enzymes including pyrrolidonyl arylamidase, α-galactosidase, β-galactosidase, alkaline phosphatase and leucine arylamidase.

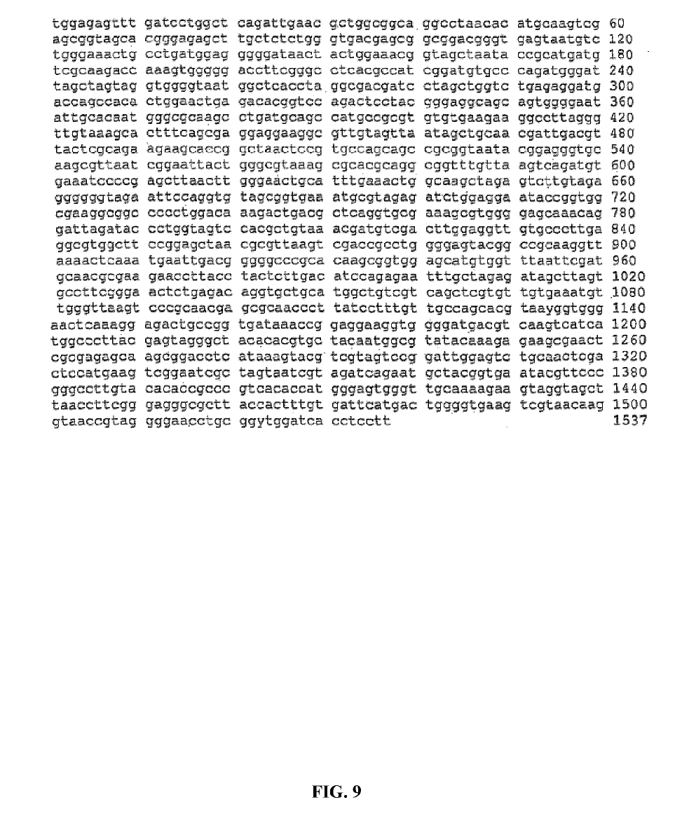

Serratia vaccinii can further be characterised as having a 16S rRNA gene sequence that comprises the nucleotide sequence as set forth in SEQ ID NO:1 (FIG. 9).

The biochemical characteristics of bacteria suitable for use in accordance with the present invention can readily be determined using standard techniques known in the art. For example, the bacterium can be identified as gram-negative by the fact it does not retain crystal violet stain in the presence of alcohol or acetone. The fermentative abilities of the bacterium can be determined, for example, by using one of a variety of kits available commercially for this purpose (for example, the API and VITEK kits from Bio Merieux, Marcy-l'Etoile, France).

It is generally accepted that a 3% variation between the 16S rRNA gene sequences of two bacteria is the point at which two strains may be considered to be separate species (see, for example, Vandamme, et al., 1996, Microbiol. Reviews 60:407-48; Kolbert & Persing, 1999, Curr. Microbiol., 2:299-305). Bacteria suitable for use in accordance with the present invention, therefore, include those with a 16S rRNA gene that comprises a sequence at least 97% identical to the sequence as set forth in SEQ ID NO:1, for example, at least 97.5% identical, at least 98% identical, at least 98.2% identical, at least 98.5% identical, at least 98.8% identical, at least 99% identical, or at least 99.5% identical to the sequence as set forth in SEQ ID NO: 1, or any amount therebetween.

Sequencing the 16S rRNA gene of a given bacterium can be readily conducted using standard DNA isolation and sequencing techniques known in the art (see, for example, Ausubel et al., Current Protocols in Molecular Biology, J. Wiley & Sons, NY) or using commercially available kits such as the MicroSeg™ 16S rRNA Gene Kit and software, available from Applied Biosystems. Comparison of the identified sequence with SEQ ID NO: 1 can be conducted using standard techniques including, for example, the use of publicly available software, such as BLAST (available from the NCBI website) and CLUSTALW (available from the EMBL-EBI website).

Serratia vaccinii can be maintained and propagated on a variety of different media using standard culture techniques at a temperature between about 8° C. and about 36° C., typically between about 10° C. and about 36° C. Examples of suitable media include, but are not limited to, tryptic soy broth or agar, Simmons citrate agar, MRS agar, Voges-Proskauer agar and potato dextrose agar.

### Fruits

Compositions in accordance with the present disclosure may be prepared using biotransformed extracts derived from one, or a mixture, of a variety of fruits. For example, grapes or various berries including, but not limited to, blueberries, cranberries, lingonberries, bilberries, blackcurrants, chokecherries, chokeberries, raspberries, blackberries, elderberries, Saskatoon berries and strawberries. In certain embodiments, the compositions comprise biotransformed extracts from one or more berries from the genus Vaccinium (including blueberries, bilberries, cranberries and lingonberries). In some embodiments, the compositions comprise biotransformed extracts from one or more of grapes, blueberries, cranberries, strawberries and Saskatoon berries. In some embodiments, the compositions comprise biotransformed extracts from one or more berries selected from blueberries, cranberries, strawberries and Saskatoon berries.

The fruit may be conventionally grown fruit, organically grown fruit or wild fruit. In certain embodiments, the fruit is organic or wild fruit.

The use of fresh, frozen, tinned or dried fruit, or combinations thereof, are contemplated in various embodiments. The fruit may be a whole fruit, or a pulp, paste, puree, juice, juice concentrate, or a solid, nectar or powdered form of the fruit. “Pulp” and “puree” refer to both heat-treated and non heat-treated whole fruit pieces, which have been mechanically transformed into soft mixture or suspension, whereas a “paste” refers to a pulp or puree that has been partially dehydrated. In some embodiments, whole, fresh fruit is used as a starting material for the preparation of the compositions. Some embodiments relate to the use of fruit juice as a starting material. In some embodiments, a powdered form of the fruit may be used as a starting material.

### Preparation of Biotransformed Fruit Extracts

Biotransformed fruit extracts can be prepared by fermentation of an appropriate fruit extract with Serratia vaccinii or a bacterium having the same identifying characteristics as Serratia vaccinii, as described above.

Certain embodiments contemplate the preparation of the biotransformed fruit extracts by fermentation of the selected fruit extract(s) with a combination of Serratia vaccinii or a bacterium having the same identifying characteristics as S. vaccinii, and a yeast. An example of a suitable yeast would be Saccharomyces cerevisae. It has been previously demonstrated that using a combination of S. vaccinii and S. cerevisae to ferment a fruit extract can improve the phenolic and thus the antioxidant content of the fermented extract (see for example, International Patent Application Publication No. 2004/101770; Vuong, T., et al., 2006, Journal of Food Biochemistry, 30:249-268). In the present disclosure, when a combination of S. vaccinii and S. cerevisae are used to ferment the selected fruit extract(s), the S. vaccinii and S. cerevisae may be used together in the same fermentation or the fruit extract may be first fermented with either S. vaccinii or S. cerevisae, and subsequently subjected to a second separate fermentation with the other organism. In certain embodiments in which a combination of S. vaccinii and S. cerevisae is employed for the fermentation, the two organisms are used together in the same fermentation.

Suitable methods of preparing biotransformed fruit extracts are described in International Patent Application Publication No. 2004/101770 and U.S. patent application Ser. No. 12/541,714 (2010/0092583). Typically, fermentation is conducted using a medium containing at least 10% (v/v) fruit extract together with sufficient amounts and proportions of ions to support bacterial growth. For example, the medium may contain at least 20%, at least 30%, at least 40%, at least 50%, at least 60%, at least 70%, at least 80% or at least 90% (v/v) fruit extract, or any amount therebetween. In certain embodiments, media may be formulated that contain up to 100% fruit extract provided that sufficient amounts and proportions of ions are incorporated to support bacterial growth. In certain embodiments, the medium may contain fruit extract and minimal media (i.e. water containing sufficient amounts and proportions of ions to support bacterial growth) in ratios (v/v) between about 9:1 and 1:9, for example between 4:1 and 1:4, between about 7:3 and 3:7, between about 3:2 and 2:3 or about 1:1.

The medium can be readily prepared, for example, by blending an appropriate amount of one or more fruits with water or a minimal medium and sterilizing the resultant solution. When the starting material is a paste, puree, dried fruit preparation or powder, the starting material may be dissolved, dispersed or reconstituted in water or minimal medium and then sterilized. Juices may be used directly or diluted in water or a minimal medium. Particulate matter can optionally be removed by standard techniques such as centrifugation or filtration. When water is used, salts and ions can be added as required. If desired, other components that promote the growth of the bacterium, such as amino acids or carbohydrates, can be incorporated into the medium. The pH of the medium can also be adjusted as necessary such that the starting pH of the medium is above 3.2. A suitable pH for the medium is in the range from about 3.3 to about 5.0, for example, between about 3.7 and about 5.0, between about 4.0 and about 5.0, or between about 4.5 and about 5.0.

Fermentation is initiated by inoculation of the medium with an appropriate number of bacterial cells as is known in the art. The inoculant can be in the form of a fraction of a starter culture, as a swab comprising cells taken from a culture of the bacteria on a solid phase, such as agar, or as a fraction of or swab from a frozen culture of the bacteria. When a starter culture is used, the starter culture can employ the same or a different medium, such as one of those described above for propagation of the bacteria.

Methods of fermentation are well-known in the art. For fermentation with Serratia vaccinii, the culture is fermented at a temperature between about 8° C. and about 36° C., for example, between about 10° C. and about 30° C., between about 15° C. and about 25° C. or between about 20° C. and about 24° C. As Serratia vaccinii is facultatively anaerobic, the fermentation can take place under aerobic or anaerobic conditions and is typically allowed to proceed for between about 1 day and about 12 days. Maximal amounts of phenolic antioxidants are usually obtained after about 2 to about 5 days of fermentation under aerobic conditions and after about 8 days of fermentation under anaerobic conditions. Accordingly, in certain embodiments, the fermentation is allowed to proceed for about 1 day to about 10 days, for example, for about 1 day to about 7 days, for about 2 days to about 5 days, for about 3 days to about 4 days or for about 4 days to about 10 days.

In some instances, the pH of the medium may vary during fermentation and this variation can affect the constitution of the fermented fruit extract. Thus, when applicable, the pH of the medium can be monitored and adjusted as necessary during the fermentation using standard techniques.

After fermentation is complete, the biotransformed extract is typically treated to remove the bacteria, although this may be optional in certain embodiments. In addition, the biotransformed extract may optionally be submitted to one or more other treatments, such as sterilization, filtration, lyophilization, purification, concentration, or the like. In certain embodiments, fermentation of the fruit extract is followed by sterilization.

### Properties of Biotransformed Extracts

Biotransformed fruit extracts may be characterized by their total phenolic content. Total phenolic content is typically measured by the Folin-Ciocalteau method using gallic acid as a standard (expressed as gallic acid equivalent (GAE)).

In certain embodiments, a fruit extract is considered to be biotransformed when the total phenolic content of the medium comprising the fruit extract used in a fermentation as described above has increased by at least 2-fold. This is referred to herein as the biotransformed fruit extract having a total phenolic content at least 2-fold greater than the corresponding untransformed extract. The fold increase in total phenolic content of the biotransformed extract over the untransformed extract can thus be measured, for example, by taking a sample of the fruit-extract containing medium prior to fermentation and a sample of the fruit-extract containing medium after fermentation and determining the GAE for each sample. If the GAE of the fermented fruit-extract containing medium is at least 2-fold greater than the GAE of the unfermented fruit-extract containing medium, then the fruit extract has been biotransformed.

Accordingly, in certain embodiments, biotransformed fruit extracts are defined as having a total phenolic content at least 2-fold greater than the corresponding untransformed extract. In some embodiments, the biotransformed extracts have a total phenolic content at least 2.5-fold greater, for example at least 3-fold greater, at least 3.5-fold greater or at least 4-fold greater than the corresponding untransformed extract. In some embodiments, the biotransformed fruit extracts have a total phenolic content at least 4-fold greater than the corresponding untransformed extract. In certain embodiments, the total phenolic acid content is defined in GAE.

### Formulations

Certain embodiments of the invention relate to formulations of the compositions for therapeutic use or cancer chemoprevention. Such formulations may be formulated as pharmaceutical compositions, or as nutraceuticals, dietary supplements, cosmetic preparations, functional foods, beverages and the like.

The formulations may be for administration for example via oral, topical, rectal or parenteral routes or for administration by inhalation or spray. The term parenteral as used herein includes subcutaneous injections, intravenous, intramuscular, intrathecal, intrasternal injection or infusion techniques. In certain embodiments, the formulations are formulated for oral administration.

Pharmaceutical compositions typically comprise one or more conventional non-toxic physiologically acceptable carriers, adjuvants or vehicles and can prepared by known procedures using well-known and readily available ingredients.

Pharmaceutical compositions may be formulated into a form suitable for oral, topical, rectal or parenteral administration, such as syrups, elixirs, tablets, troches, lozenges, hard or soft capsules, pills, suppositories, oily or aqueous suspensions, dispersible powders or granules, emulsions, injectables, or solutions.

Nutraceutical formulations also may be formulated, for example, as syrups, elixirs, tablets, troches, lozenges, hard or soft capsules, pills, suppositories, oily or aqueous suspensions, dispersible powders or granules or emulsions, as well as teas, tonics, juices, syrups, bars, or the like.

Cosmetic formulations may be formulated, for example, as lotions, gels, creams, ointments, foams, oils, or sprayable liquids.

Formulations intended for oral use may in some embodiments be prepared in either solid or fluid unit dosage forms. Fluid unit dosage form can be prepared according to procedures known in the art for the manufacture of pharmaceutical compositions and such compositions may contain one or more agents selected from sweetening agents, flavouring agents, colouring agents, preserving agents, and the like, in order to provide pharmaceutically elegant and palatable preparations. An elixir is prepared by using a hydroalcoholic (for example, ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Solid formulations such as tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or talc and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein active ingredient(s) are mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the active ingredient(s) with an acceptable vegetable oil, light liquid petrolatum or other inert oil.

Aqueous suspensions contain active ingredient(s) in admixture with excipients suitable for the manufacture of aqueous suspensions. Such excipients are suspending agents, for example sodium carboxylmethylcellulose, methyl cellulose, hydroxypropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl-p-hydroxy benzoate, one or more colouring agents, one or more flavouring agents or one or more sweetening agents, such as sucrose or saccharin.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Formulations may also be in the form of oil-in-water emulsions. The oil phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. Adjuvants such as local anaesthetics, preservatives and buffering agents can also be included in the injectable solution or suspension.

For local administration to the eye, the formulations can be formulated for administration by eye drops, injection or the like. In the case of eye drops, the formulation can also optionally include, for example, ophthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

In certain embodiments, the compositions may be formulated together with other extracts, for example herbal extracts or extracts from fruits or vegetables, that have beneficial properties in cancer management, prevention or treatment or that provide other health benefits to the patient.

Other formulations and methods of preparing same are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

### Efficacy of the Compositions

A number of standard tests to determine the anti-cancer properties of a compound or composition are known in the art and can be employed to test the compositions and/or formulations. Initial determinations of the efficacy of the compositions may be made using one or more standard in vitro assays. Exemplary procedures are described in the Examples provided herein. Other standard in vitro assays known in the art may also be employed.

The ability of the compositions to inhibit tumour growth, proliferation and/or metastasis in vivo can be determined in an appropriate animal model using standard techniques known in the art (see, for example, Enna, et al., Current Protocols in Pharmacology, J. Wiley & Sons, Inc., New York, N.Y.). Inhibition of CSC development may also be tested using standard techniques, such as those described in the Examples provided herein.

In addition, the compositions can optionally be submitted to other standard tests, such as cytotoxicity tests, stability tests, bioavailability tests and the like. As will be readily apparent to one skilled in the art, the compositions will need to meet certain criteria in order to be suitable for human or animal use and to meet regulatory requirements. Thus, once a composition has been found to be suitable for animal administration, standard in vitro and in vivo tests can be conducted to determine information about the metabolism and pharmacokinetics (PK) of the compositions, including data on drug-drug interactions where appropriate, which can be used to design human clinical trials. Toxicity and dosing information can likewise be obtained through standard pre-clinical evaluations. Appropriate dosages can be readily determined from such pre-clinical data and, when necessary, the compositions can be evaluated for their efficacy in standard clinical trials procedures.

### Uses

The compositions comprising one or more biotransformed fruit extracts as described herein may have application in various aspects of cancer treatment, management and/or prevention. In this context, the compositions may be incorporated into formulations as described above, for example, pharmaceuticals, nutraceuticals, dietary supplements, cosmetics, functional foods, beverages, and the like, for use in the treatment, management and/or prevention of cancer. Accordingly, the compositions may be used in various embodiments by subjects having cancer, subjects at risk of having cancer, and/or healthy subjects.

As demonstrated in the Examples, biotransformed blueberry juice is capable of influencing components of key signaling pathways involved in tumour invasion and metastasis, and in the development of cancer stem cells (CSCs). For example, biotransformed blueberry juice was shown to increase phosphorylation of the MAPK members p38 MAPK and JNK and to decrease phosphorylation of ERK1/2 in cancer cells. The biotransformed blueberry juice also inhibited activation of the IL-6 pathway in cancer cells and was demonstrated to affect the expression of a number of cancer-related miRNAs, including increasing expression of the tumour-suppressor miRNA, miR-145, and decreasing expression of miR-210 miRNA, which is involved in cancer growth and invasion. These components are known to play important roles in a broad spectrum of cancer types. In addition, the biotransformed juice inhibited tumour growth, tumour metastasis and CSC development in various breast cancer cell lines, as well as melanoma cell lines and prostate cancer cell lines. Thus, in certain embodiments, it is contemplated that the compositions described herein will have broad therapeutic applicability for a number of different cancers.

Certain embodiments of the invention relate to methods of using the compositions comprising biotransformed fruit extract(s) to inhibit activation of the IL-6 pathway in cancer cells. IL-6-dependent pathways can enhance tumour growth and refractoriness to chemotherapy. Accordingly, in some embodiments, the compositions may find use in decreasing tumour growth and/or the development of chemoresistance.

In some embodiments, the compositions may be used in methods of inhibiting miR-210 expression and/or increasing miR-145 expression in cancer cells. miR-210 and miR-145 may play important roles in regulation of tumouor cell growth, angiogenesis and apoptosis.

Certain embodiments of the invention relate to the use of the compositions to inhibit cancer metastasis in a subject, for example metastasis of primary tumours. In some embodiments, the compositions may be administered to a subject having an early stage cancer to help attenuate the progression of the disease through their effect on tumour growth and/or metastasis. The latter effect is particularly useful in further slowing down a cancer that progresses relatively slowly, such as prostate cancer. In some embodiments, the compositions may be administered to a patient prophylactically to attenuate the growth or metastasis of a tumour. This application is particularly useful for those patients having an aggressive disease that is known to metastasise readily. In some embodiments, the compositions may be administered to a patient prophylactically to prevent the development of a cancer.

Certain embodiments of the invention relate to the use of the compositions to inhibit cancer stem cell (CSC) development. CSCs have been implicated in a variety of cancers, including but not limited to the following cancers: brain, breast, colon, ovary, pancreas, prostate, melanoma, and multiple myeloma. The presence of CSCs in a tumour can contribute to relapse of the cancer, therapy resistance, metastasis and progression. Accordingly, certain embodiments of the invention relate to the use of the compositions to delay or prevent relapse, to treat drug-resistant cancers and/or aggressive cancers, to inhibit the development of drug-resistance, to inhibit metastasis, and/or delay or prevent progression. In certain embodiments, the invention relates to the use of the compositions in a cancer selected from brain cancer, breast cancer, colon cancer, ovarian cancer, pancreatic cancer, prostate cancer, liver cancer, bladder cancer, melanoma, and multiple myeloma.

Retinoblastoma tumours have also been shown to contain a small subpopulation of cells that exhibit a cancer stem cell-like phenotype (Seigel, G. M., et al., 2005, Mol Vis., 11:729-37). Accordingly, certain embodiments of the invention contemplate the use of the compositions in the treatment or prevention of retinoblastoma.

Certain embodiments of the invention relate to the use of the compositions in cancer chemoprevention, for example, to lower the risk of a subject developing cancer or to slow cancer development in a subject. The use of the compositions in cancer chemoprevention may be particularly useful, for example, in subjects who have a higher risk of developing cancer, such as those with a previous cancer, an inherited cancer syndrome, or a family history of cancer. Certain embodiments of the invention relate to the use of the compositions for cancer chemoprevention in a subject having or at risk of developing a cancer selected from brain cancer, breast cancer, colon cancer, ovarian cancer, pancreatic cancer, prostate cancer, liver cancer, bladder cancer, melanoma, and multiple myeloma. Some embodiments relate to the use of the compositions for cancer chemoprevention in a subject having or at risk of developing breast cancer.

Certain embodiments of the invention relate to the use of the compositions to delay or prevent the recurrence of a cancer in a subject. In some embodiments, the compositions may be administered to a patient as part of a secondary therapy regimen to delay recurrence or relapse and/or prolong survival. “Secondary therapy” refers to a therapeutic regimen started after a primary therapy, such as surgery or radiation.

Certain embodiments of the invention relate to the use of the compositions to treat cancers resistant to conventional therapies, such as drug-resistant cancers. In some embodiments, the compositions may be used to inhibit the development of drug-resistance in a tumour. In this context, the compositions may be used, for example, as a adjunct therapy to a conventional chemotherapeutic regimen.

In some embodiments, the invention relates to methods of delaying or preventing the progression of a cancer in a subject by administering a composition comprising a biotransformed fruit extract. In some embodiments, the invention relates to methods of improving the efficacy of conventional cancer therapies, such as chemotherapy or radiation, by administering to a subject a composition comprising a biotransformed fruit extract. In some embodiments, the invention relates to methods of using a composition comprising a biotransformed fruit extract in adjunct therapy for the treatment of cancer.

Certain embodiments of the invention relate to the use of the compositions in combination with one or more anti-cancer therapeutics with the intention of improving the efficacy of the anti-cancer therapeutic(s), for example as part of an adjunct therapy. In this context, the compositions may result in a decrease the amount of the anti-cancer therapeutic required to achieve the desired effect and thereby lead to an increased efficacy, decreased side-effects and/or more cost-effective treatment regimens. Alternatively, this approach can be taken in the treatment of drug-resistant cancers unresponsive to standard treatment in order to weaken the tumour with the intention of rendering it susceptible to standard therapeutics. The compositions may also be used in this context to potentiate the effect of standard doses of the anti-cancer therapeutic, or to potentiate to effect of sub-optimal doses of the anti-cancer therapeutic in those patients who cannot tolerate standard doses.

Thus, certain embodiments of the invention contemplate administration to a subject of a composition as described herein together with one or more anti-cancer therapeutics. The composition may be administered before, during or after treatment with the anti-cancer therapeutic. An “anti-cancer therapeutic” is a compound, composition or treatment that prevents or delays the growth and/or metastasis of cancer cells. Such anti-cancer therapeutics include, but are not limited to, chemotherapeutic drug treatment, radiation, gene therapy, hormonal manipulation, and biologic therapy including immunotherapy and antisense oligonucleotide therapy. In certain embodiments, the compositions may also be used with standard combination therapies employing two or more anti-cancer agents.

Certain embodiments relate to the use of the compositions as a complementary therapy in the treatment of cancer. In this context, the compositions may be taken by a patient with cancer to augment traditional therapies. For example, the compositions may be formulated as a nutraceutical or health supplement to be taken by a cancer patient either prophylactically to reduce the likelihood that cancer stem cell development, metastasis and/or progression will occur, or therapeutically to decrease cancer stem cell development, metastasis and/or progression that is already underway.

In certain embodiments, the compositions are formulated as a natural health product, supplement, beverage or cosmetic for prophylactic use to help prevent the development of cancer, or progression or worsening of an existing cancer. Certain embodiments of the invention thus relate to the use of the composition as a supplement, for example, in the form of a pill, tablet or capsule, or as a tea or other beverage, for prophylactic administration. Some embodiments relate to the use of the compositions as a nutraceutical that may be taken alone or added to a foodstuff as a prophylactic.

Some embodiments relate to the use of the compositions as an ingredient in a cosmetic for skin health, for example, to help prevent the development of cancer from exposure to environmental factors such as UV.

To gain a better understanding of the invention described herein, the following examples are set forth. It will be understood that these examples are intended to describe illustrative embodiments of the invention and are not intended to limit the scope of the invention in any way.

### EXAMPLES

The following Examples demonstrate the superior anti-cancer activity of blueberry juice biotransformed by fermentation with Serratia vaccinii when compared to untransformed blueberry juice. The anti-cancer effects of blueberries that have been previously observed have been attributed to the polyphenol content of these fruits (see, for example, Adams L. S., et al., 2010, ibid.). Biotransformed blueberry juice is known to have an increased polyphenol content (Martin, L. and Matar, C., 2005, ibid.). However, a more recent study (Montales, M. T., et al., 2012, ibid.) demonstrated that a mixture of isolated blueberry polyphenols limited activity to only one of the two cancer cell lines tested and higher concentrations showed a decreased activity, suggesting that high polyphenol content could in fact result in a restricted applicability in cancer types as well as a reduced activity. Surprisingly, as shown herein, biotransformed blueberry juice, even when administered at the same total phenolic content (as measured by gallic acid equivalents (GAE)) as untransformed blueberry juice, showed both higher levels and a more broadly applicable anti-cancer activity than the untransformed blueberry juice, indicating that the anti-cancer activities of the biotransformed juice are likely a result of unknown compounds resulting from the fermentation and/or synergistic interaction of multiple components in the biotransformed juice.

In addition, it is believed that the biotransformation process may have led to long polyphenol chains, which are poorly absorbed in gastro-intestinal tracts, being broken down into shorter more bioavailable chains thus increasing the ability of these compounds to exert an effect on the tumours. Large quantities of long chain polyphenols have also been shown to have an undesirable pro-oxidant effect, which would be avoided by the breakdown of the compounds into shorter chains by the biotransformation process.

Statistical Analysis.

Statistical analysis of the data by ANOVA and Bonferroni's post-hoc tests were performed using GraphPad Prism software (San Diego, Calif., USA). Statistical significance was set at p≦0.05. Data are reported as mean±SEM.

### Example 1

### Inhibition of Breast Cancer Cell Proliferation

Preparation of blueberry juices. Mature lowbush blueberries (Vaccinium angustifolium Ait.) were purchased from Cherryfield Foods Inc. (Cherryfield, Me., USA) as fresh and untreated fruits. Blueberry juice was extracted by blending the fruit (100 g) and minimal medium (100 mL) in a Braun Type 4259 food processor. The fruit mixture was then centrifuged at 500×g for 10 min to remove fruit skin and insoluble particles. The resulting juice was sterilized using 0.22 μm Express Millipore filters (Millipore, Etobicoke, Ontario, Canada).

Serratia vaccinii bacteria were cultured as previously described (Martin, L. and Matar, C., 2005, J Sci Food Agri, 85:1477-1484). The juice was inoculated with a saturated culture of Serratia vaccinii corresponding to 2% of the total juice volume. After a four-day fermentation period, the transformed juice was sterilized by 0.22 um filtration. The total phenolic content was measured by the Folin-Ciocalteau method using gallic acid as standard and hence expressed as μM Gallic Acid Equivalent (GAE). The total phenolic content was increased from 5.9 mM GAE to 30.7 mM GAE, confirming successful transformation. Blueberry juice and biotransformed blueberry juice (BBJ) have been partially characterized elsewhere (Martin, L. and Matar, C., 2005, ibid.; Matchett, M. D., et al., 2006, J Nutr Biochem, 17:117-125).

Cell Culture.

Murine 4T1, human MCF-7 and human MDA-MB-231 cell lines were obtained from American Type Cell Collection (ATCC; Chicago, Ill.). MCF-7 cells were cultured in MEM, 4T1 and MDA-MB-231 in RPMI-1640, media containing FBS (10%, v/v) (Sigma-Aldrich, Oakville, ON, Canada), penicillin/streptomycin (0.05 mg/mL) at 37° C. in a humidified atmosphere with 5% CO₂.

Cell Viability.

Cell viability was assessed by WST-1 and LDH assays (Roche, Laval, QC, Canada). After a 24 h treatment, supernatants were collected for LDH assay following the manufacturer's instructions. Cells were washed once with RPMI-1640 and WST-1 was added (10%, v/v) and incubated at 37° C. for 2 h. The absorbance was measured at 450 nm in a μ-Quant plate reader (Bio-Tek, Winooski, Vt.).

Cell Proliferation Results.

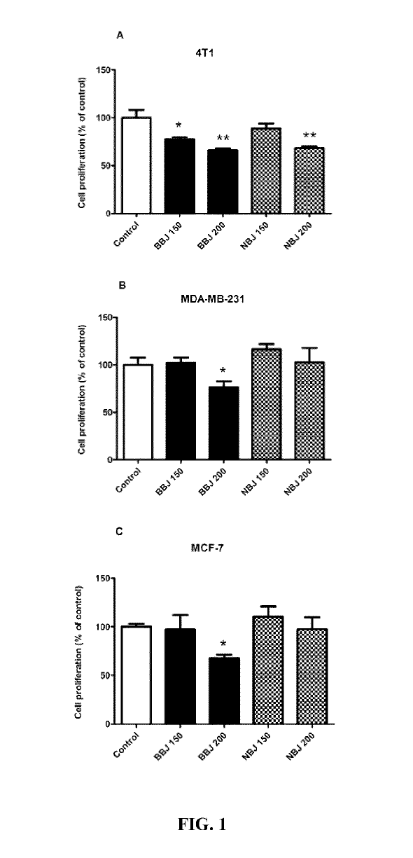

At a concentration of 200 μM GAE, biotransformed blueberry juice (BBJ) significantly inhibited the proliferation of 4T1, MDA-MB-231 and MCF-7 cancer cells by 34%, 24% and 33% respectively (FIG. 1), whereas untransformed juice (NBJ) showed an inhibition of 32% in 4T1 cells proliferation only (FIG. 1, panel A). Hence, the antiproliferative effect of BBJ was observed in all of three breast cancer cell lines at 200 μM whereas NBJ, at the same concentration, only had an effect in 4T1. No significant effects of NBJ were observed in MDA-MB-231 and MCF-7 (FIG. 1, panel B-C).

### Example 2

### Reduction of Metastatic Potential

Cell Lines.

Murine 4T1, human MCF-7 and human MDA-MB-231 cell lines were obtained from American Type Cell Collection and cultured as described in Example 1.

Cell Mobility.

Cells were plated in a six-well plate at a concentration of 1×10⁶ per well and allowed to form a confluent monolayer for 24 h. Cells were then serum starved for 24 hours, and the monolayer was scratched with a pipette tip, washed with RPMI-1640 to remove floating cells, and photographed (time 0). Cells were treated with NBJ or BBJ (prepared as described in Example 1) for 24 or 48 h. Cells were then photographed again at three randomly selected sites per well. The migrated cell surface area was expressed as percent of closure.

Cell Invasion.

The cell invasion assay was performed on a polyethylene terephthalate (PET) membrane (8-m pore size) in a Tissue Culture (TC) insert (BD Biosciences, Mississauga, ON). Cells were plated into the upper chamber of the insert containing serum-free medium (SFM), and the insert was placed into a well of a 24-well plate containing complete medium with the presence/absence of various concentrations of NBJ and BBJ. After 24 hours, the top surface of the TC insert was scraped using a cotton swab and the cells on the lower surface of the membrane were incubated for 1 hour with Calcein AM. The plate was read in a fluorescence plate reader at excitation wavelength of 485 nm and emission wavelength of 550 nm at a gain of 55 nm. Data are expressed as a ratio to the control group.

Results.

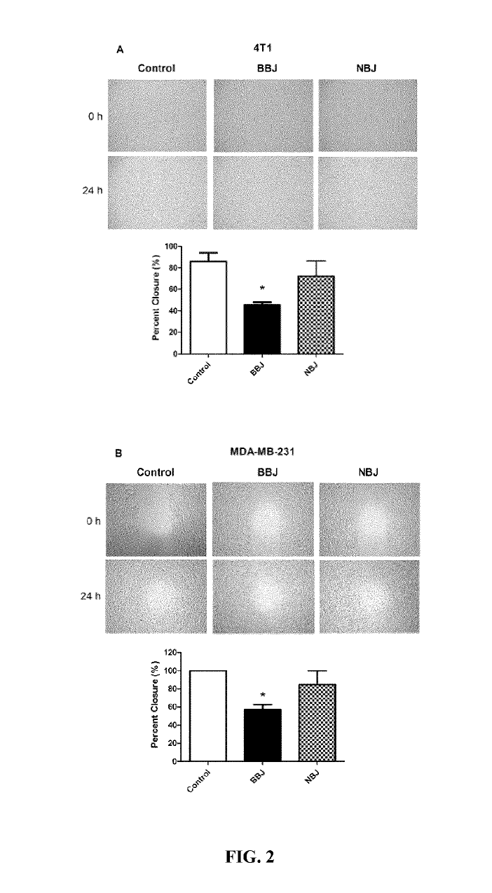

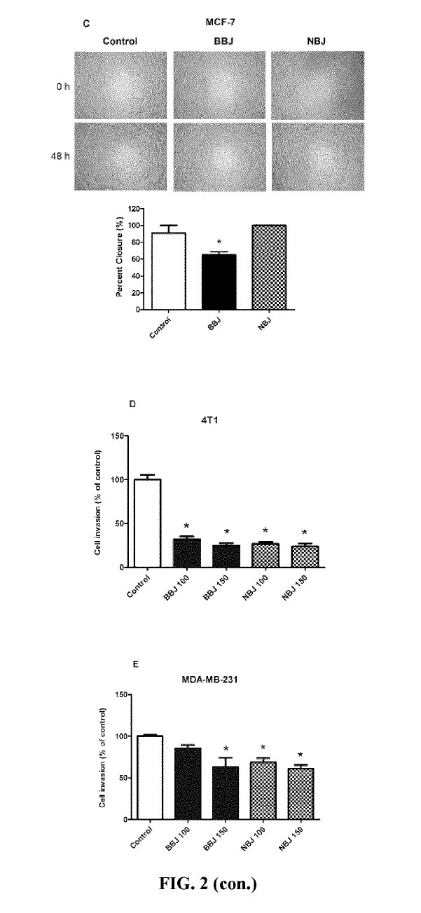

Both NBJ and BBJ at 150 M GAE significantly reduced the invasive ability of 4T1 and MDA-MB-231 (FIG. 2, panels D and E). However, only BBJ exhibited an inhibitive effect on the motility of all three breast cancer cell lines (FIG. 2, panels A-C). NBJ did not show any significant effect on cell motility as compared to the control.

### Example 3

### Inhibition of Mammosphere Formation

Cell Lines.

Murine 4T1, human MCF-7 and human MDA-MB-231 cell lines were obtained from American Type Cell Collection and cultured as described in Example 1.

Mammosphere Formation.

Adherent cells were detached by trypsin and single cells were counted using Countess (Invitrogen). For tumour tissue, approximately 0.05 g of each tumour was minced and dissociated in RPMI-1640 media containing 300 U/ml collagenase (#C7657, Sigma), and 100 U/ml hyaluronidase (#H3631, Sigma) at 37° C. for 2 h. Cells were sieved sequentially through a 100 μm and a 40 μm cell strainer (BD Biosciences) to obtain a single cell suspension, and counted in a haemocytometer. Single cells were plated in ultralow attachment 96-well plates (#3474, Costar) at 10³ cells/0.2 ml/well, in the presence/absence of BBJ and NBJ (prepared as described in Example 1) in DMEM-F12 (#12660, Invitrogen), supplemented with 10 ng/ml EGF, 20 ng/ml bFGF, 5 μg/ml insulin, 1 mM sodium pyruvate, 0.5 μg/ml hydrocortisone, and penicillin/streptomycin (0.05 mg/mL). Cells grown in these conditions as non-adherent spherical clusters of cells or mammospheres were counted after 4-7 days.

Results.

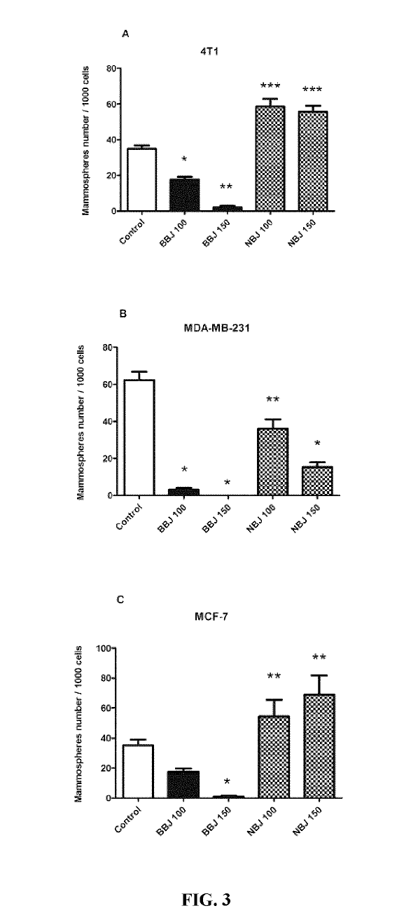

BBJ significantly decreased the formation of mammospheres in all three cell lines (FIG. 3), and total inhibition was observed at 150 μM GAE of BBJ. Treatment with the same concentration of NBJ exhibited an inhibition of 75% in MDA-MB-231 only (FIG. 3, panel B), whereas it significantly increased the formation of mammospheres in 4T1 and MCF-7 by 60% and 96%, respectively (FIG. 3, panel A and C). The inhibition of mammosphere formation by BBJ demonstrates its anti-metastatic properties. In addition, as the formation of mammospheres is a characteristic of cancer stem cell (CSC) development (Charafe-Jauffret, E., et al., 2009, Cancer Res, 69:1302-1313; Polytarchou, C., et al., 2012, Proc Natl Acad Sci USA, 109:14470-14475), this Example is also indicative of the ability of BBJ to inhibit CSC development.

### Example 4

### Deregulation of IL-6-Related Pathways

Cell Lines.

Murine 4T1, human MCF-7 and human MDA-MB-231 cell lines were obtained from American Type Cell Collection and cultured as described in Example 1.

IL-6 Determination.

BD OptEIA Mouse IL-6 ELISA sets (BD Biosciences, Mississauga, ON) were used to measure IL-6 production following the manufacturer's instructions.

Results.

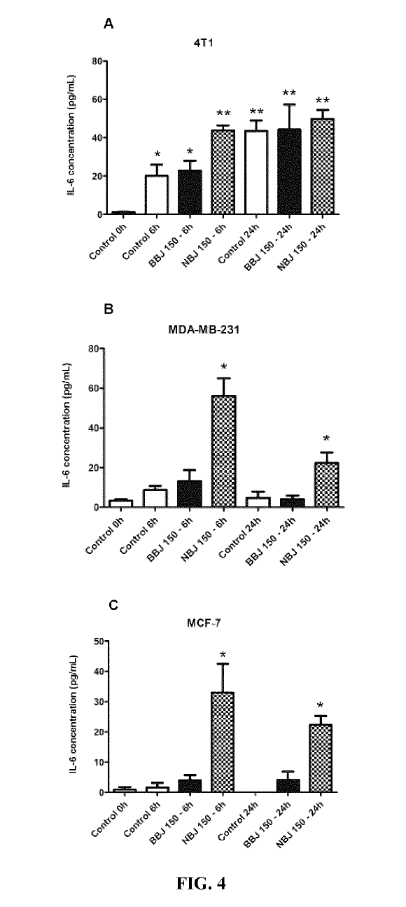

A 6 h-treatment with NBJ in mammosphere formation conditions significantly elevated the secretion of IL-6 in all three cell lines (FIG. 4), while BBJ (prepared as described in Example 1) did not induce any modification as compared to control cells (FIG. 4).

High serum levels of IL-6 correlate with poor outcome in breast cancer patients (Bachelot, T., et al., 2003, Br J Cancer, 88:1721-1726). Recent studies indicated that deregulation of IL-6 signaling induces malignant features in mammary stem/progenitor cells (Iliopoulos, D., et al., 2009, Cell, 139:693-706; Sansone, P., et al., 2007, J Clin Invest, 117:3988-4002). Identified as breast cancer stem cells (Al-Hajj, M., et al., 2003, Proc Natl Acad Sci USA, 100:3983-3988), they are able to self-renew, produce differentiated progeny, and migrate into surrounding tissues (Stingl, J. and Caldas, C., 2007, Nat Rev Cancer, 7:791-799; Visvader, J. E. and Lindeman, G. J., 2008, Nat Rev Cancer, 8:755-768). They enhanced metastasis, resistance to cancer treatment, and relapse (Sharma, A., et al., 2012, Mol Cancer Ther, 11:77-86; Korkaya, H., et al., 2012, Mol Cell, 47:570-584). Tumour formation and persistent self-renewal observed in cancerous stem cells were reported to be epigenetically controlled by deregulation of DNA methylation at the promoter of genes involved in IL-6 signalling pathway (Hernandez-Vargas, H., et al., 2012, Epigenetics, 6:428-439). Thus, prevention and/or inhibition of deregulation in IL-6 signalling pathway could be beneficial for the treatment and better outcome of breast cancer.

### Example 5

### Effect on IL-6 and MAPKs Pathways

Cell Lines.

Murine 4T1, human MCF-7 and human MDA-MB-231 cell lines were obtained from American Type Cell Collection and cultured as described in Example 1.

Cell Signalling by Milliplex.

After treatments, cells were collected and lysed. Cell lysates were analyzed using 9-Plex Multi-Pathway MAG Panel kits on a MagPix apparatus (#48-608MAG, Millipore, Etobicoke, Ontario, Canada) following the manufacturer's instructions.

Western Blot Analysis.

Cells were treated in the presence/absence of BBJ and NBJ, in mammospheres formation conditions as above then collected and lysed after 1, 2, 6 and 24 h. Cell lysates were run on a 10% acrylamide gel, transferred to a PVDF membrane, and probed with either anti-phosphorylated PI3K, PDK1, PTEN, p38 MAPK, ERK1/2, SAPK/JNK, (cat #4228, #3438, #9551, #9211, #4377, #9251 respectively, Cell Signaling Tech. Inc., Danvers, Mass., USA). Bands were visualized via chemiluminescence using horseradish peroxidase-conjugated secondary antibodies. Bands were quantified using Bio-Rad Quantity One software.

Inhibition of IL-6 Signalling Pathway.

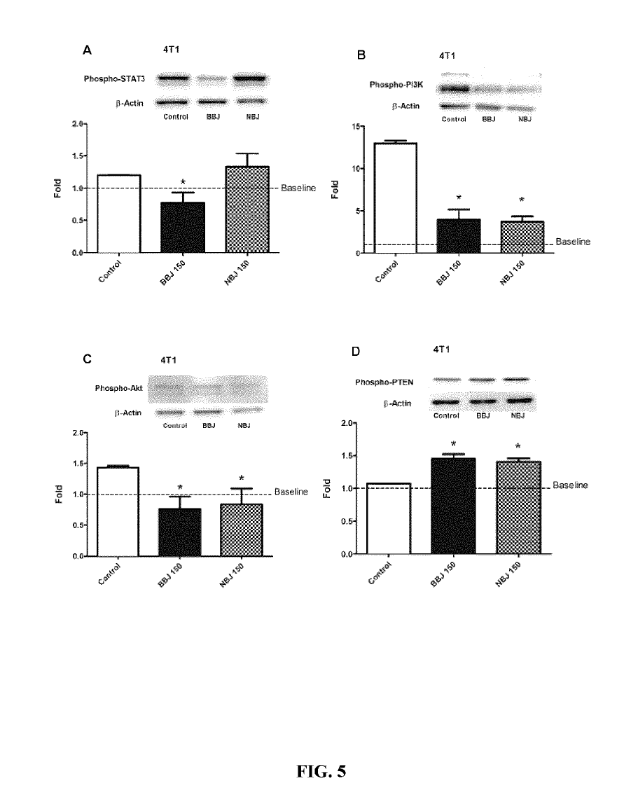

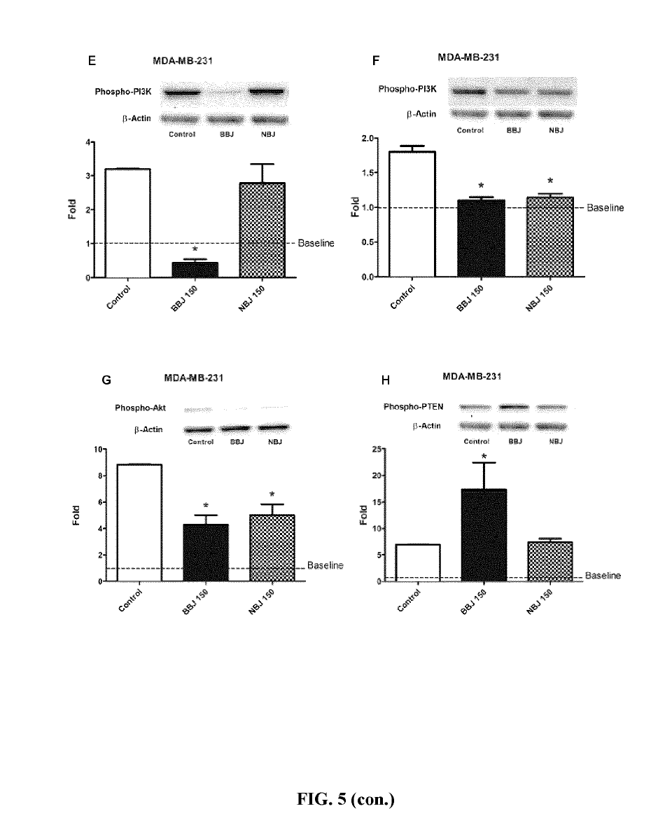

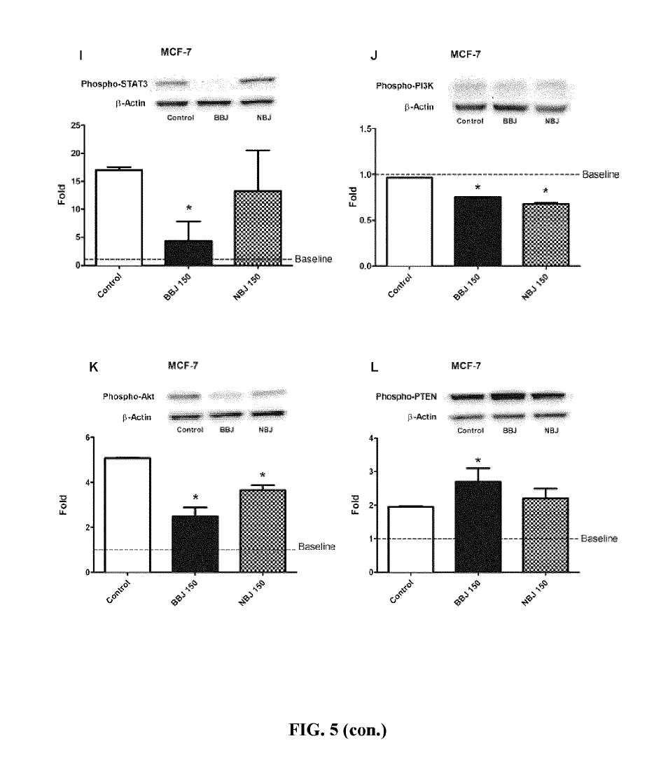

BBJ (prepared as described in Example 1) significantly inhibited the phosphorylation of STAT3 and PI3K/Akt in all three cell lines. This inhibition started after a 6 h-treatment (FIG. 5, panels A-C, E-G and I-K) and lasted up to 24 h, whereas NBJ decreased the phosphorylation of PI3K only. Both BBJ and NBJ significantly enhanced the activity of PTEN in 4T1 (FIG. 5, panel D) but only BBJ increased PTEN phosphorylation in MDA-MB-231 and MCF-7 (FIG. 5, panels H and L).

Alterations of MAPKs Pathway.

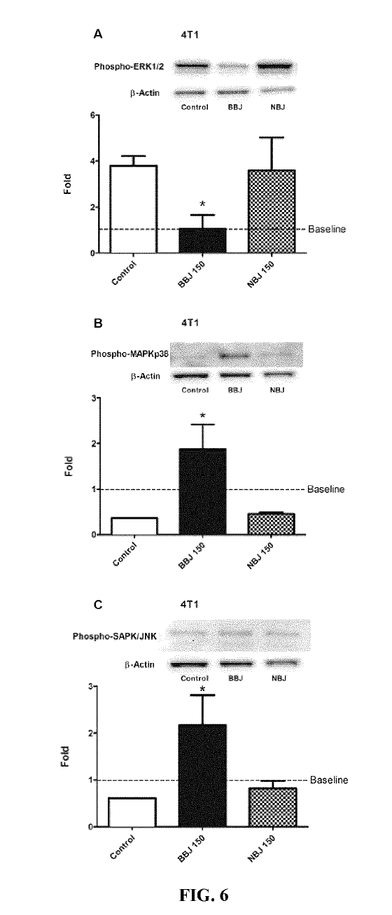

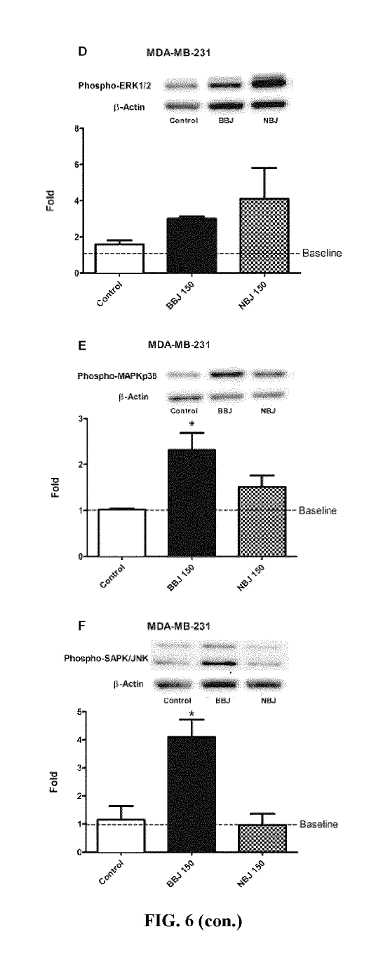

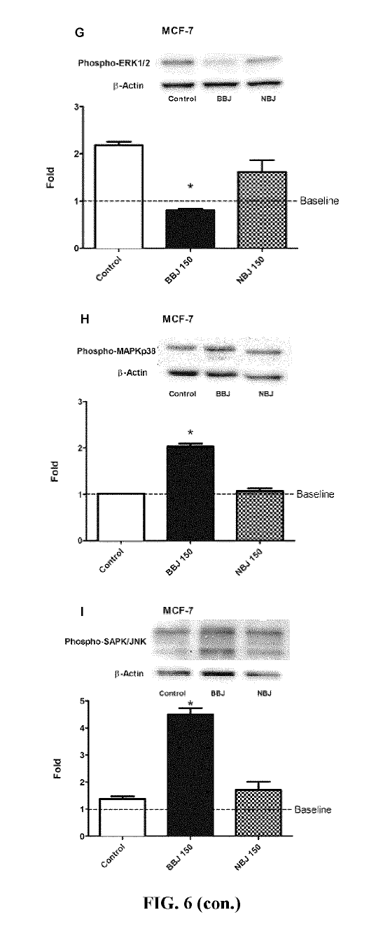

Starting one hour after the addition of BBJ, a significant inhibition of ERK1/2 phosphorylation was observed in 4T1 and MCF-7 (FIG. 6, panels A and G). BBJ also increased MAPK p38 and JNK/SAPK phosphorylation in all three cell lines (FIG. 6, panels B, C, E, F, H and I). The maximal inhibition or activation in these cells was attained after 2 h of treatment and remained stable up to 24 h. NBJ did not show any significant modification of the tested MAPK family members.

The reduced activities of three distinct members of the IL-6 pathway, STAT3/PI3K/PDK1, in the presence of BBJ support a negative feedback proposal. The IL-6 transcription factor STAT3 has been recently recognized as the key target to reduce tumour growth and angiogenesis (Lamy, S., et al., 2012, Exp Cell Res, 318:1586-1596) and metastasis (Zhao, X., et al., 2012, Asian Pac J Cancer Prev, 13:2873-2877) in different types of cancer. This STAT-3 inhibition could be negatively correlated with the development of breast cancer stem cells. Diet-derived polyphenols have been reported to inhibit the IL-6/STAT3 signaling at three different check points of this pathway (Lamy, S., et al., 2012, ibid.). Firstly, they could reduce the gene expression of IL-6 receptor such as IL-6α (Lamy, S., et al., 2012, ibid.). Secondly, they down-regulated the active tyrosin-phosphorylated forms of JAK1 and JAK2 (Weissenberger, J., et al., 2012, Clin Cancer Res, 16:5781-5795), or inhibited the phosphorylation of STAT3 (Weissenberger, J., et al., 2012, ibid.; Wang, X., et al., 2012, Int J Oncol, 40:1189-1195). Finally, they enhanced the expression of JAK/STAT3 inhibitors, such as Suppressor of cytokine signaling (SOCSs) (Lamy, S., et al., 2012, ibid.) and Protein inhibitor of activated STAT (PIASs) (Saydmohammed, M., et al., 2010, J Cell Biochem, 110:447-456).

Both BBJ and NBJ could inhibit the phosphorylation of PI3K. These findings are consistent with previous reports, which attributed the inhibition of PI3K activity to the anticancer effects of blueberry (Adams, L. S., et al., 2010, ibid.; Montales, M. T., et al., 2012, Carcinogenesis, 33:652-660). In this Example, BBJ and NBJ were also shown to enhance the activity of PTEN, an upstream inhibitor protein of PI3K, probably via the inhibition of miRNA-21 expression (Liu, Z. L., et al., 2012, Mol Cell Biochem, 372(1-2):35-45).

Additionally, BBJ significantly inhibited ERK1/2 in CSCs in a non-cell type manner (FIG. 6). In MAPK pathways, ERK1.2 us the most relevant to breast cancer. Increased ERK1/2 was recently reported as driving endocrine resistance and breast cancer progression in an obesity-related experimental model (Bowers, et al., 2013, Breast Cancer Res., 15:R59).

Although 4T1, MDA-MB-231 and MCF-7 have been widely used to study anticancer properties of polyphenols, detailed information on the signaling pathways involved during stem cell growth is still lacking, notably at the level of the three branches of the MAPK family, namely ERK1/2, JNK, and p38 MAPK. In this Example, the kinetics of these members of the MAPK family were investigated in the presence of polyphenols from blueberry juice during the development of breast cancer stem cells. This does not appear to have been reported previously in these cell systems.

NBJ was without effect on the three members of the MAPK family and results were generally similar to the respective controls. In contrast, treatment with BBJ rapidly increased p38 MAPK and JNK phosphorylations, with significant differences observed after one hour, with the highest level reached at 2 h, and elevated levels maintained for up to 24 h. Conversely, BBJ reduced ERK1/2 phosphorylation in the same kinetic manner. Modifications in MAPK family enzymes appear to contribute to the abolition of stem cell growth afforded by BBJ. Indeed, prolonged activation of JNK and MAPKp38 and/or inhibition of ERK1/2 results in induction of apoptosis in most cancer cell lines (Chen, J. and Sun, L., 2012, Horm Metab Res, 44(13):943-8; Yang, L. H., et al., 2012, Mol Med Report, 6:1126-1132; Na, H. K., et al., 2012, Biochem Pharmacol, 84(10):1241-50; Leisner, T. M., et al., 2012, Oncogene, epub.). The mechanisms by which BBJ modified MAPK activities are unknown. BBJ potentially acts as a weak oxidant, and activates ROS-MAPKs pathways, the same mechanism as anticancer drugs (Yip, N. C., et al., 2011, Br J Cancer, 104:1564-1574; Gopalan, A., et al., 2012, Cancer Lett, 329(1):9-16). Another possible explanation relates to the epigenetic hypothesis since MAPKs are encoded by miR-145 and miR-543, which have been reported as tumour suppressors capable of inhibiting cell growth, invasion and metastasis in breast cancer cells (Feifei, N., et al., 2012, J Cancer Res Clin Oncol, 138(11):1937-44).

Apoptosis is still one of the major mechanisms against cancer stem cell progression (Huang, M., et al., 2012, Mol Biotechnol, 45:39-48). In fact, it is well known that chemotherapeutic drugs induce cellular damage, and thus drive cancer cells into apoptosis (O'Connor, M. J., et al., 2007, Oncogene, 26:7816-7824). Many of the commonly used chemotherapeutic drugs exhibit some selectivity for tumour cells via members of the MAPK pathways (Yip, N. C., et al., 2011, ibid.; Chen, J., et al., 2012, Anticancer Drugs, 23:98-107). ERK1/2 has been linked to cell proliferation and survival, whereas the stress-activated MAPKs, p38 and JNK have been connected with apoptosis (Wagner, E. F. and Nebreda, A. R., 2009, Nat Rev Cancer, 9:537-549). Therefore, chemotherapy could be enhanced via modulations of these three members of the MAPK pathway.

The investigations described in this Example demonstrated that only BBJ could activate the MAPK cascade and is therefore likely impacting on the IL-6 pathway. These alterations are most likely exerted by novel compounds produced during biotransformation of the blueberry juice as they were not observed with NBJ. Moreover, there might be an extensive cross-talk and interplay between MAPK cascade and IL-6 pathway since the inhibition of STAT3/PI3K/PDK1 occurred after the activation of MAPK members in this study. Indeed, the spatial distribution and temporal qualities of MAPKs can markedly alter the qualitative and quantitative features of downstream signaling to immediate early genes (IEG) and the expression of IEG-encoded protein products. As a result, IEG products provide a molecular interpretation of MAPK dynamics, enabling the cell to program an appropriate biological response (Murphy, L. O. and Blenis, J., 2006, Trends Biochem Sci, 31:268-275).

### Example 6

### Reduction of Tumour Growth, Mammosphere Formation and Metastasis In Vivo

Animals.

Six- to 8-week-old BALB/c female mice weighing 18-20 g (Charles River, Montreal, QC) were randomly distributed into seven experimental groups: control, NJ12.5, NJ25, NJ50, BJ12.5, BJ25 and BJ50. Each experimental group consisted of 8 mice housed in a controlled atmosphere (temperature 22±2° C.; humidity 55±2%) with a 12 h light/dark cycle. Mice were maintained and treated in accordance with the guidelines of the Canadian Council on Animal Care. While mice in the control group received normal water, mice in NBJ- and BBJ-groups received either NBJ or BBJ (prepared as described in Example 1), incorporated into their drinking water at three concentrations: 12.5%, 25% and 50% (v/v) respectively. After a two-week treatment, all mice received a subcutaneous injection of 4T1 cells (1400 cells/0.1 ml/mice) into the abdominal mammary gland fat pad. Three weeks after the inoculation, tumours and lungs were collected and weighed. Both BBJ and NBJ were well tolerated and did not affect the body weight of the treated mice.

Mammosphere Formation.

For tumour tissue, approximately 0.05 g of each tumour was minced and dissociated in RPMI-1640 media containing 300 U/ml collagenase (#C7657, Sigma), and 100 U/ml hyaluronidase (#H3631, Sigma) at 37° C. for 2 h. Cells were sieved sequentially through a 100 μm and a 40 μm cell strainer (BD Biosciences) to obtain a single cell suspension, and counted in a haemocytometer. Single cells were plated in ultralow attachment 96-well plates (#3474, Costar) at 10³ cells/0.2 ml/well, in DMEM-F12 (#12660, Invitrogen), supplemented with 10 ng/ml EGF, 20 ng/ml bFGF, 5 μg/ml insulin, 1 mM sodium pyruvate, 0.5 g/ml hydrocortisone, and penicillin/streptomycin (0.05 mg/mL). Cells grown in these conditions as non-adherent spherical clusters of cells or mammospheres were counted after 4-7 days.

Lung Metastasis.

Lungs obtained from the mice were minced and dissociated in RPMI-1640 media containing 300 U/ml collagenase (#C7657, Sigma), at 37° C. for 15 min. After filtration through a 40 μm cell strainer (BD Biosciences), cells were collected and suspended in RPMI-1640 containing 10% FBS (ATCC), penicillin/streptomycin (0.05 mg/mL) and 60 μM 6-thioguanine 60 (Sigma). Cells were plated in 10-cm culture dishes (Corning) at 37° C. in a humidified atmosphere with 5% CO₂. After 14 days, cells were fixed with methanol and stained with 0.03% methylene blue solution. All blue colonies were counted, one colony representing one clonogenic metastatic cell.

Results.

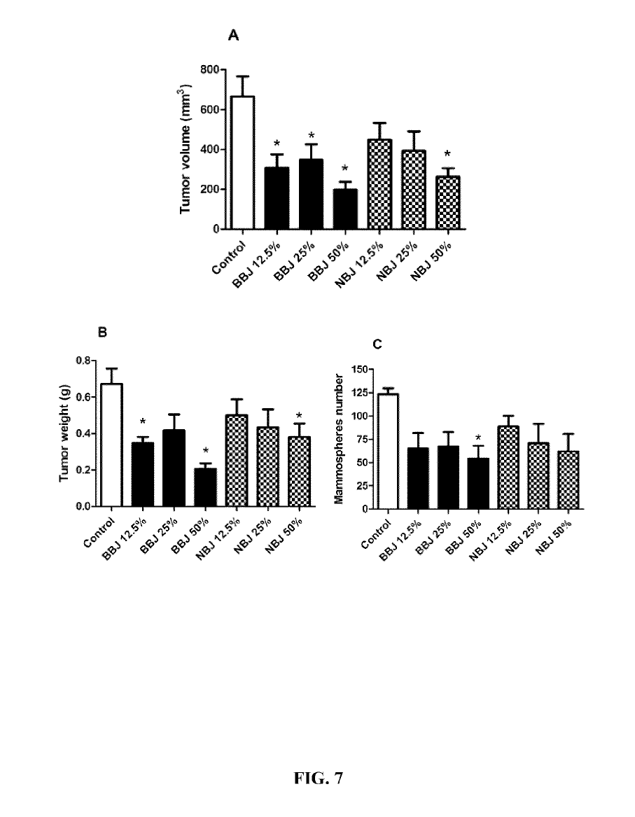

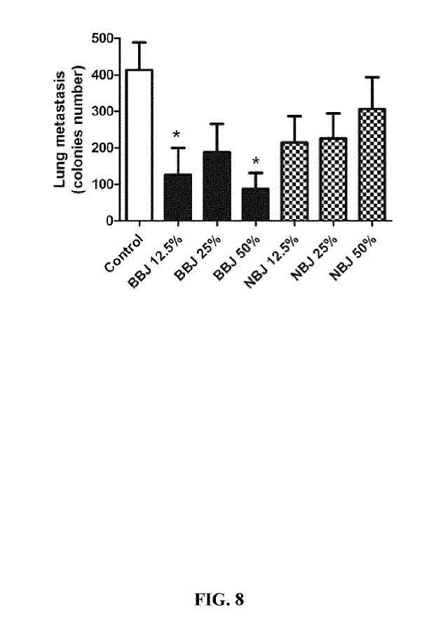

As illustrated in FIG. 7, when administered chronically over a 5-week period, NBJ reduced tumour volume and weight in a dose-dependent manner. However, for the groups of mice that received NBJ, significant effects were observed only in the NJ50 group, whereas all three dose levels of BBJ significantly delayed tumour development in BBJ-treated-mice (FIG. 7, panel A). Moreover, mammosphere formation from tumoural primary cells was significantly reduced only in tumours of BJ50-treated animals (FIG. 7, panel C). Similarly, only BBJ-treatment could significantly reduce metastasis in the lungs of the mice, while all of the other groups did not show a significant difference as compared to control animals (FIG. 8).

These results demonstrate the in vivo anti-cancer and anti-metastatic potential of BBJ using the 4T1-induced breast cancer model in BALB/c mice. The 4T1 tumour has several characteristics that make it a suitable experimental animal model for human mammary cancer. Firstly, tumour cells are easily transplanted into the mammary gland so that the primary tumour grows in the anatomically correct site. Secondly, the 4T1 tumour is highly tumourigenic and invasive and, unlike most tumour models, can spontaneously metastasize from the primary tumour in the mammary gland to multiple distant sites including lymph nodes, blood, liver, lung, brain, and bone as in human mammary cancer (Pulaski, B. A. and Ostrand-Rosenberg, S., 1998, Cancer Res, 58:1486-1493; Lelekakis, M., et al., 1999, Clin Exp Metastasis, 17:163-170). The results convincingly showed that BBJ holds great promise as an anti-cancer and anti-metastatic agent.

Chronic administration of BBJ via incorporation into the drinking water of the treated mice significantly reduced tumour volume and breast cancer stem cell development inside the tumour. This reduction supports and explains the low count of metastasis in the lungs of BBJ-treated animals. BBJ anti-cancer and anti-metastatic effects were observed at a therapeutic dose as low as 12.5%, which corresponds to approximately 1.2 cups of juice per day in humans. In contrast, normal blueberry juice at the same dose did not show any significant effect. Only at the high dose of 50%, which corresponds to approximately 5 cups of juice per day in humans, was a decrease in tumour size and weight observed in NBJ-treated animals. While these results are consistent with findings from previous studies which reported feeding blueberry extracts or whole fruit powder could delay tumour growth in mice (Adams, L. S., et al., 2010, ibid.; Montales, M. T., et al., 2012, ibid.), it is important to note that NBJ failed to achieve the reduction of breast cancer stem cells and metastasis observed with BBJ.

BBJ has been shown to have a much higher content of total phenolic compounds than NBJ, which could contribute to its effectiveness at low therapeutic dose as compared to NBJ. However, the anti-metastatic activity of BBJ is more likely explained by the change of phenolic composition from NBJ to BBJ during the biotransformation process. Indeed, the biotransformation of blueberry juice not only increases its phenolic content but also produces novel compounds (Martin, L. and Matar, C., 2005, ibid.). Given that BBJ was used at the same Gallic Acid Equivalent (GAE) as NBJ, one interesting possibility is that these novel compounds may possess more potent anticancer and antimetastatic properties that could have contributed to reduction in tumour size and metastasis observed with BBJ administration. In addition, the biotransformation process may have led to long polyphenol chains, which are poorly absorbed in gastro-intestinal tracts, being broken down into shorter more bioavailable chains thus increasing their ability to exert an effect on the tumours.

### Example 7

### Inhibition of Skin Cancer Cell Proliferation

Cell Culture.

Murine B16F10 and human HS294t cell lines were obtained from American Type Cell Collection (ATCC; Chicago, Ill.). Cells were cultured in DMEM media containing FBS (10%, v/v) (Sigma-Aldrich, Oakville, ON, Canada), penicillin/streptomycin (0.05 mg/mL) at 37° C. in a humidified atmosphere with 5% CO₂.

Cell Viability.

Cell viability was assessed by WST-1 and LDH assays (Roche, Laval, QC, Canada). After a 24 h treatment, supernatants were collected for LDH assay following the manufacturer's instructions. Cells were washed once with RPMI-1640 and WST-1 was added (10%, v/v) and incubated at 37° C. for 2 h. The absorbance was measured at 450 nm in a t-Quant plate reader (Bio-Tek, Winooski, Vt.).

Cell Proliferation Results.

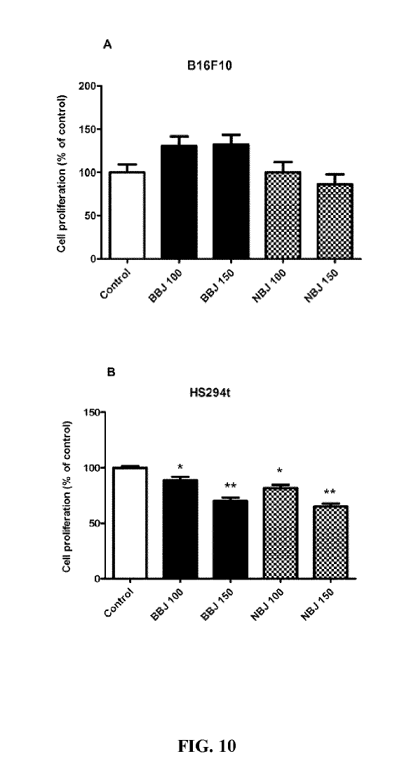

Neither BBJ and NBJ (prepared as described in Example 1) showed any significant effects on the proliferation of murine B16F10 skin cancer cells (FIG. 10, panel A). However, both BBJ and NBJ significantly inhibited the proliferation of human HS294t cancer cells by 12% and 30% at 100 μM and 150 μM GAE, respectively (FIG. 10, panel B). No significant difference between BBJ and NBJ were observed at each concentration tested. Thus, direct treatment of skin cancer cells in vitro with NBJ and BBJ significantly inhibits cell proliferation of melanoma HS294T.

### Example 8

### Inhibition of Spheroid Formation in Melanoma Cells

Spheroid formation.

Adherent cells were detached by trypsin and single cells were counted using Countess (Invitrogen). Single cells were plated in ultralow attachment 96-well plates (#3474, Costar) at 10³ cells/0.2 ml/well, in the presence/absence of BBJ and NBJ (prepared as described in Example 1), in DMEM-F12 (#12660, Invitrogen), supplemented with 10 ng/ml EGF, 20 ng/ml bFGF, 5 g/ml insulin, 1 mM sodium pyruvate, 0.5 g/ml hydrocortisone, and penicillin/streptomycin (0.05 mg/mL). Cells grown in these conditions that formed non-adherent spherical clusters of cells or spheroids were counted after 5-7 days.

Inhibition of Spheroid Formation.

BBJ significantly decreased the formation of spheroids in both B16F10 and HS294t cell lines in a dose-dependent manner (FIG. 11). At 100 μM GAE concentration, BBJ reduced the formation of spheroids in both cell lines by 72% and 84%, whereas NBJ did not have any significant effect at this concentration. Only at 150 μM GAE concentration, NBJ showed a slight but significant inhibition in both cell lines (FIG. 11). Thus, only BBJ showed significant inhibition of spheroid formation in both cell lines at low concentration.

### Example 9

### Effect on miRNA Expression

MicroRNAs (miRNAs) represent a subset of endogenous small non-coding RNAs with a striking ability to control the expression of approximately one third of the human genome. These small, non-coding RNAs could inhibit target genes expression by binding to the 3′ untranslated region of target mRNA, resulting in either mRNA degradation or inhibition of translation. They are often over-expressed or down-regulated in a number of malignancies and some can also function as tumor suppressors or as oncogenic agents.

A global miRNA array analysis was conducted in mammosphere cultures of mammary carcinoma 4T1 to examine the effect on miRNA expression after treatment with BBJ. Total RNA was purified using a TRIzol (Invitrogen)/miRNeasy Mini Kit (Qiagen) from 4T1 cells treated with BBJ and NBJ for 1, 6 and 24 hours. 1,000 ng of RNA was tagged using Affymetrix FlashTag™ Biotin HSR (Affymetrix, Inc., Santa Clara, Calif., USA). miRNA analysis was performed using an Affymetrix GeneChip miRNA 3.0 Array on a Genechip 7G Scanner (Affymetrix).

The results for a selection of modulated miRNAs are shown in Table 1.

TABLE 1
Selection of Over- and Under-Expressed miRNAs in 4T1 Cells after
Treatment with BBJ*

| | |
|---|---|
| Over-expressed | Under-expressed |

| miRNA | Fold change | miRNA | Fold change |
|---|---|---|---|
| miR-145 | 3.04 | miR-7 | 0.37 |
| miR-34b | 2.13 | miR-450 | 0.40 |
| miR-26a | 1.97 | miR-23b | 0.44 |
| miR-216b | 1.95 | miR-214 | 0.46 |
| miR-101 | 1.86 | miR-210 | 0.51 |
| let-7g | 1.77 | miR-301 | 0.52 |
| miR-150 | 1.73 | miR-297 | 0.54 |
| miR-365 | 1.70 | miR-30e | 0.57 |
| miR-195 | 1.65 | miR-182 | 0.55 |
| miR-146a | 1.48 | | |

*Data are expressed as “fold change” over untreated cells at 0 h.

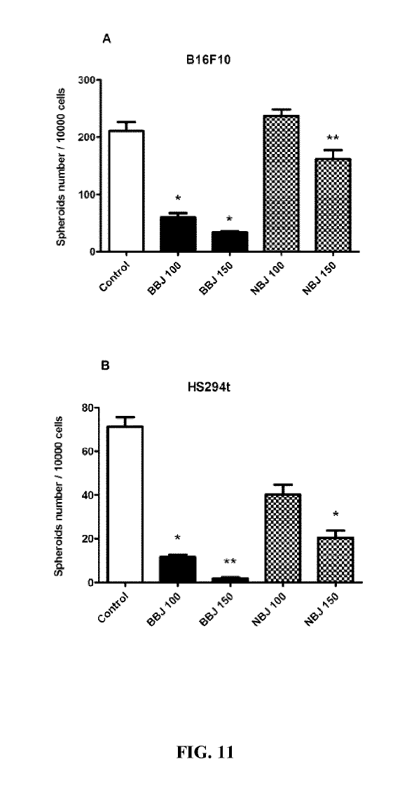

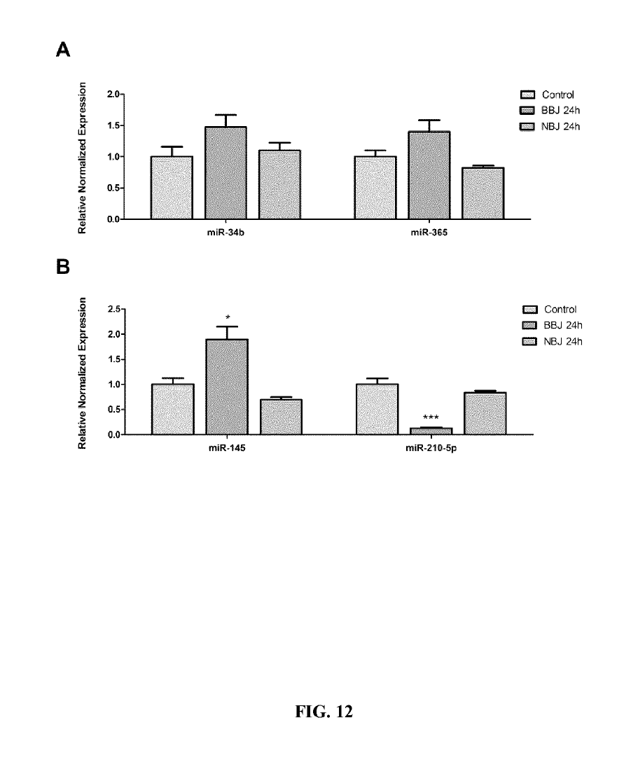

Subsequent qRT-PCR analysis of the expression of selected microRNA by 4T1 cells after a 24 hour treatment with 60 mEG/ml of biotransformed blueberry juice (BBJ) or normal blueberry juice (NBJ) is shown in FIG. 12.

The results revealed that several miRNAs associated with different clinical pathologic characteristics of breast cancer such as sternness, invasion and chemoresistance were differentially expressed (Table 1). In particular, miR-210, the most consistently and robustly induced miRNA under hypoxia was found to be highly down-regulated (FIG. 12). MiR-210 is over-expressed in a variety of human tumors and in cancer cell lines in hypoxic conditions, a key feature of the tumor microenvironment. Specifically, miR-210 was shown to decrease proapoptotic signaling in a hypoxic environment, suggesting an impact on tumor formation, and invasion. Interestingly, miR-210 expression was correlated with metastasis of breast and melanoma tumors. This observation aligns with the results of the preceding Examples in which BBJ is demonstrated to decrease the formation of CSCs in different types of melanomas and mammary carcinoma cell lines. A very recent study reported that hypoxia leads to increased expression of VEGF, IL-6, and CSC signature genes such as Nanog, and Oct4 with increased cell migration/invasion, concomitant with increased expression of miR-210 in human pancreatic cancer cells.

The results also revealed an over-expression of miR-26a, a miRNA belonging to hypoxia miRNA group (Table 1). Down-regulation of miR-26a is found to be associated with poor prognosis of hepatocellular carcinoma and correlated with recurrence. IL-6 was identified as a target of miR-26a, and miR-26a dramatically suppressed expression of STAT3 target genes including Bcl-2, Mcl-1, cyclin D1, and MMP2. This is consistent with the results in the preceding Examples which show that BBJ down-regulated STAT3.

Five to six miRNAs that have been shown to be associated with IL-6 pathways were affected by BBJ (Table 1); miR-365, let-7g, miR-146a, miR-145 and miR-26a. One of the most over-expressed miRNAs is miR-365, a novel negative regulator of IL-6 and known tumour suppressor. MiR-145, an AKT-cancer-associated miRNA that is assumed to play a role in invasion, is also over-expressed in BBJ-treated cells. MiR-145 has been found to usually be under-expressed in breast cancer with high metastatic capability. In prostate cancer, miR-145 is down-regulated in primary cancer compared with normal prostate tissue, and is associated with bone metastasis and gleason score (Zaman, C et al. 2010, Br J Cancer 103(2):256-264). MiR-146a was also found to significantly regulate IL-6 and iNOS in human glial cells. Since BBJ-induced modulation of the PTEN/PI3K/AKT pathway was also accompanied by a decrease of STAT3 (see preceding Examples), BBJ control of miR-145 could potentially lead to tumour control and regression. MiR-145 is also regulated by AKT in p53-dependent manner. Suppression of PI3K activity substantially increases p53 levels and at the same time induces miR-145 (Sachdeva, Z et al. 2009, Proc Natl Acad Sci USA, 106(9):3207-3212). In particular, p53 appears to up-regulate the expression of 1) tumor suppressor miRNAs such as let-7, miR-34, miR-145, miR-26, miR-30, and miR-146a; 2) tumor suppressor genes such as PTEN, RBs, CDKN1, and 3) metastasis suppressors such as Raf kinase inhibitory protein, CycG2, thereby inhibiting tumorigenesis, invasion, metastasis, and CSC proliferation. Over-expression of let-7g and miR-195, tumor suppressors that inhibit invasion and metastasis, was also observed in BBJ-treated cells (Table 1).

MiR-34b has been shown to be silenced in human prostate cancer (Wang et al. 2013, Int J Oncol 42(3):957-962) and was over-expressed in BBJ-treated cells. Functionally, miR-34b over-expression inhibited cell proliferation, and migration/invasion, by directly targeting the AKT and decreasing tumour growth in nude mice (Majid, D et al. 2013, Clin Cancer Res 19(1):73-84).

Taken together, the results in the preceding Examples suggest that BBJ induces epigenetic changes by modulating miRNA regulatory networks, resulting in a genetic reprogramming and inhibition of CSCs-dependent survival/stemness pathways.

### Example 10

### Effect on Spheroid Formation in Prostate Cancer Cell Lines

Prostatosphere Formation:

LNCaP and DU145 prostate cancer cells were detached by trypsin and single cells were counted using Countess (Invitrogen). Single cells were plated in ultralow attachment 96-well plates (#3474, Costar) at 10³ cells/0.2 ml/well, in the presence/absence of BBJ and NBJ (prepared as described in Example 1), in DMEM-F12 (#12660, Invitrogen), supplemented with 10 ng/ml EGF, 20 ng/ml bFGF, 5 μg/ml insulin, 1 mM sodium pyruvate, 0.5 μg/ml hydrocortisone, and penicillin/streptomycin (0.05 mg/mL). Cells grown in these conditions as non-adherent spherical clusters of cells (or prostatospheres) were counted after 5 days.

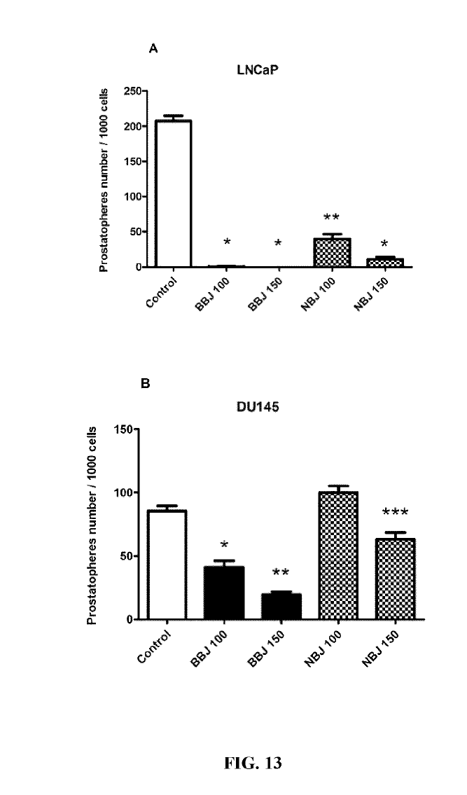

BBJ significantly decreased the formation of prostatospheres in both cell lines (FIG. 13). In LNCaP cells, a total inhibition was observed at 100 μM GAE of BBJ as compared to an inhibition of 80% in NBJ-treated cells. Treatment with the same concentration of NBJ in DU145 cells resulted in a slight and but non-significant increase of prostatosphere formation. Only at 150 μM GAE, NBJ showed a significant inhibition of 26%, whereas BBJ significantly inhibited the formation of prostatospheres by 52% and 77%, at 100 and 150 μM GAE, respectively (FIG. 13).

### Example 11

### Effect on Phosphorylation of STAT3, Akt and p70S6K in Prostate Cancer Cell Spheroids

Phosphorylation of STAT3, Akt and p70S6K in the prostatospheres isolated in Example 10 was analyzed.

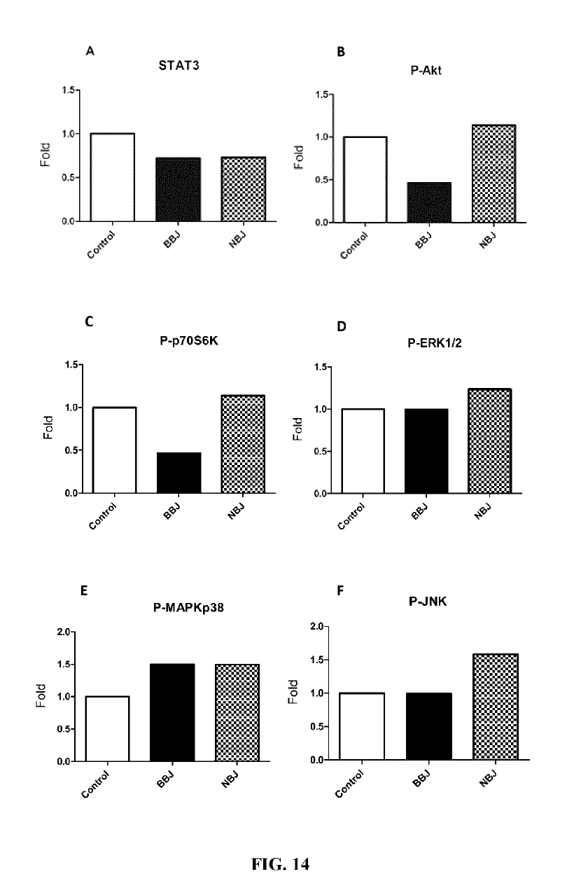

BBJ significantly inhibited the phosphorylation of STAT3, Akt and p70S6K (FIG. 14A-C). This inhibition started after a 6 h-treatment and lasted up to 24 h, whereas NBJ only decreased the phosphorylation of STAT3.

### Example 12

### Effect on Phosphorylation of ERK1/2, MAPKp38, and JNK in Prostate Cancer Cell Spheroids

Phosphorylation of ERK1/2, MAPKp38 and JNK in the prostatospheres isolated in Example 10 was analyzed.

While BBJ was observed to increase the phosphorylation of MAPK p38 only in the experiment shown in FIG. 14D-F, inhibition of ERK1/2 was observed in one experiment. The ability of BBJ to inhibit ERK1/2 in prostate cancer cells is expected to be confirmed in subsequent experiments given that inhibition was observed in 4T1 cells.

NBJ showed a potent activation of all three MAPK family members (FIG. 14D-F).

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1.-45. (canceled)

46. A method of inhibiting cancer stem cell development in a subject comprising administering to the subject an effective amount of a composition comprising a biotransformed fruit extract, wherein the biotransformed fruit extract is prepared by fermenting a fruit extract with a bacterial strain having all the characteristics of Serratia vaccinii.

47. (canceled)

48. The method according to claim 46, wherein the inhibition of cancer stem cell development inhibits cancer metastasis in the subject.

49.-50. (canceled)

51. The method according to claim 46, wherein the cancer is brain cancer, breast cancer, colon cancer, ovarian cancer, pancreatic cancer, prostate cancer, liver cancer, bladder cancer, melanoma or multiple myeloma.

52. The method according to claim 46, wherein the cancer is breast cancer, prostate cancer or melanoma.

53. The method according to claim 46, wherein the fruit extract is from one or more berries from the genus Vaccinium.

54.-57. (canceled)

58. The method according to claim 46, wherein the biotransformed fruit extract is characterized as having a total phenolic content at least two times greater than untransformed fruit extract.

59. The method according to claim 46, wherein the composition is administered orally to the subject.

60. The method according to claim 46, wherein the composition is formulated as a pharmaceutical composition, a nutraceutical, a dietary supplement, a cosmetic preparation, a functional food or a beverage.

61.-71. (canceled)

72. A method of complementary therapy for the treatment of cancer comprising administering to a subject undergoing an anti-cancer treatment an effective amount of a composition comprising a biotransformed fruit extract, wherein the biotransformed extract is prepared by fermenting a fruit extract with a bacterial strain having all the characteristics of Serratia vaccinii.

73. The method according to claim 72, wherein the cancer is brain cancer, breast cancer, colon cancer, ovarian cancer, pancreatic cancer, prostate cancer, liver cancer, bladder cancer, melanoma or multiple myeloma.

74. The method according to claim 72, wherein the cancer is breast cancer, prostate cancer or melanoma.

75.-79. (canceled)

80. The method according to claim 72, wherein the biotransformed fruit extract is characterized as having a total phenolic content at least two times greater than untransformed fruit extract.

81. The method according to claim 72, wherein the composition is administered orally to the subject.

82. The method according to claim 72, wherein the composition is formulated as a pharmaceutical composition, a nutraceutical, a dietary supplement, a cosmetic preparation, a functional food or a beverage.

83. A method of decreasing the risk of a subject developing cancer comprising administering to the subject an effective amount of a composition comprising a biotransformed fruit extract, wherein the biotransformed extract is prepared by fermenting a fruit extract with a bacterial strain having all the characteristics of Serratia vaccinii.

84. The method according to claim 82, wherein the cancer is brain cancer, breast cancer, colon cancer, ovarian cancer, pancreatic cancer, prostate cancer, liver cancer, bladder cancer, melanoma or multiple myeloma.

85. The method according to claim 82, wherein the cancer is breast cancer, prostate cancer or melanoma.

86. The method according claim 82, wherein the fruit extract is from one or more berries from the genus Vaccinium.

87.-93. (canceled)

94. A method for inhibiting expression of miR-210 miRNA in a cancer cell comprising contacting the cancer cell with a composition comprising a biotransformed fruit extract, wherein the biotransformed extract is prepared by fermenting a fruit extract with a bacterial strain having all the characteristics of Serratia vaccinii.


SEQUENCE LISTING (1 sequence)

SEQ ID NO: 1 - DNA, 1537 bp, Serratia vaccinii
tggagagtttgatcctggctcagattgaacgctggcggcaggcctaacacatgcaagtcgagcggtagcacgggagagcttgctctctgggtgacgagcggcggacgggtgagtaatgtctgggaaactgcctgatggagggggataactactggaaacggtagctaataccgcatgatgtcgcaagaccaaagtgggggaccttcgggcctcacgccatcggatgtgcccagatgggattagctagtaggtggggtaatggctcacctaggcgacgatcctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgtagttaatagctgcaacgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcacgtaayggtgggaactcaaaggagactgccggtgataaaccggaggaaggtggggatgacgtcaagtcatcatggcccttacgagtagggctacacacgtgctacaatggcgtatacaaagagaagcgaactcgcgagagcaagcggacctcataaagtacgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggytggatcacctcctt